(19) United States
(12) Patent Application Publication
(10) Pub. No.: US 2023/0136215 A1
(43) Pub. Date: May 4, 2023

(54) PRODUCTION OF DUNALIELLA

(71) Applicant: University of Greenwich, London (GB)

(72) Inventors: Patricia Janine Harvey, London (GB); Yanan Xu, London (GB)

(21) Appl. No.: 17/964,312

(22) Filed: Oct. 12, 2022

Related U.S. Application Data
(62) Division of application No. 16/763,919, filed on May 13, 2020, now abandoned.

(30) Foreign Application Priority Data
Nov. 14, 2017 (GB) 1718822.8
Nov. 23, 2017 (GB) 1719440.8
Aug. 23, 2018 (GB) 1813776.0

(51) Int. Cl. A61K 36/05; A23L 33/105; A61K 8/9722; C12N 1/12

(52) U.S. Cl. CPC A61K 36/05; A23L 33/105; A61K 8/9722; C12N 1/12

(57) ABSTRACT

The present invention provides Dunaliella alga, and extracts thereof, comprising increased levels of 9-cis-β-carotene and/or increased levels of colourless carotenoids; and/or increased levels of α-carotene, to processes for producing such Dunaliella alga, and to uses thereof.

### CROSS-REFERENCE TO RELATED APPLICATIONS

The present application is a divisional of U.S. patent application Ser. No. 16/763,919, filed May 13, 2020, the entire contents of which are hereby incorporated herein by reference; U.S. patent application Ser. No. 16/763,919 is a National Stage of International Application No.: PCT/GB2018/053278 that was filed Nov. 13, 2018; the entire contents of which are hereby incorporated by reference; International Application No. PCT/GB2018/053278 claims priority to GB patent application No. 1718822.8 that was filed Nov. 14, 2017, GB patent application No. 1719440.8 that was filed Nov. 23, 2017, and GB patent application No. 1813776.0 that was filed Aug. 23, 2018 the entire contents of all three of which are incorporated herein by reference.

### TECHNICAL FIELD

The present invention relates to Dunaliella algae, and extracts thereof, comprising increased levels of 9-cis β-carotene and/or increased levels of colourless carotenoids and/or increased levels of α-carotene, to processes for producing such Dunaliella algae, and to uses thereof.

### BACKGROUND

Dunaliella is a green alga which is known to produce high concentrations of β-carotene, a naturally occurring pigment which has a variety of uses, including as a food colourant, an additive for cosmetics, and a nutritional or health supplement for veterinary and human use.

Other natural sources of β-carotene include carrots and palm oil, however, these produce a significantly lower β-carotene content compared with Dunaliella algae. D. salina is considered the best commercial source of natural β-carotene in the world (Borowitzka M.; J. Appl. Phycol. 1995; 7:3-15). β-Carotene exists in the all-trans, and in the 9-cis forms with the known natural sources producing β-carotene predominantly as the all-trans isomer. Synthetic methods for the production of β-carotene provide exclusively the all-trans isomer and there is no known method of converting all-trans-β-carotene to 9-cis-β-carotene. OsD27, a 9-cis/all-trans β-carotene isomerase, catalyses the reversible isomerization between 9-cis- and all-trans β-carotene but conversion of 9-cis into all-trans β-carotene is the preferred reaction (Bruno, M. & Al-Babili, S., 2016, Planta, 243(6), pp. 1429-1440).

Chemical Structures of A) 9-cis-β-carotene and B) all-trans-β-carotene

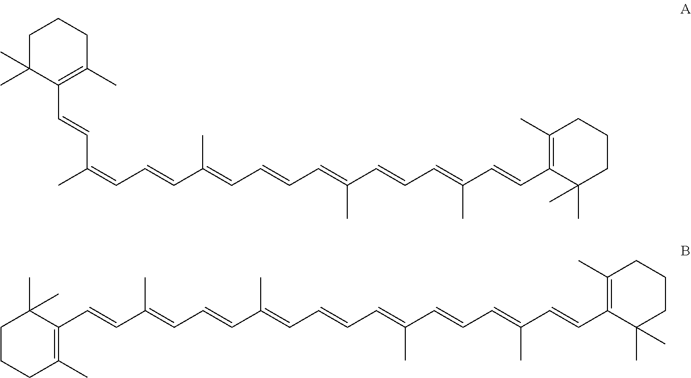

Therapeutic uses of Dunaliella salina bardawil have been proposed: US 2010/0221348 A1 discusses the use of Dunaliella salina bardawil powder in the treatment of atherosclerosis and/or diabetes mellitus. Shaish et al (The alga Dunaliella: physiology, genomics and biotechnology, ISBN 1578085454) hypothesize that the beneficial effects of Dunaliella salina bardawil on atherosclerosis is due to its high content of 9-cis-β-carotene. A clinical trial to test the effect of Dunaliella salina bardawil on psoriasis is discussed in Greenberger et al (J. Am. Coll. Nutr., 2012, October, 31(5), 320-326). Trials investigating the effect of Dunaliella salina bardawil on retinal dystrophy and retinitis pigmentosa are discussed in Rotenstreich et al (Br. J. Opthalmol., 2010, May, 94(5), 616-621 and JAMA Opthalmol., 2013, August, 131(8), 985-92).

The major all-trans isomer has low solubility in aqueous solvent systems and tends to form crystals or precipitate, requiring formulation in oil based systems or emulsions, and thus limiting the industrial and clinical utility of all-trans β-carotene. The minor 9-cis β-carotene has been found to dissolve crystalline all-trans β-carotene and to reduce the tendency of the all-trans form to precipitate. It would therefore be an advantage to produce β-carotene comprising predominantly the 9-cis isomer, which β-carotene can be more easily formulated. However, extraction of natural β-carotene from Dunaliella followed by purification to increase the ratio of 9-cis: all-trans β-carotene is currently the only known commercial method for producing preparations with a high 9-cis β-carotene content, as discussed in U.S. Pat. No. 5,612,485 and European Patent Application No. EP0933359. A recent paper Sher et al, 2018 (Scientific Reports (2018) 8:6130) discusses a synthetic method for the preparation of 9-cis-beta-carotene.

Dunaliella algae are also known to produce significant concentrations of the colourless carotenoids phytoene (IUPAC name (6E,10E,14E,16Z,18E,22E,26E)-2,6,10,14,19,23,27,31-octamethyldotriaconta-2,6,10,14,16,18,22,26,30-nonaene) and phytofluene (IUPAC name (6E,10E,12E,14E,16E,18E,22E,26E)-2,6,10,14,19,23,27,31-octamethyldotriaconta-2,6,10,12,14,16,18,22,26,30-decaene), precursors in the biosynthesis of all carotenoids. Phytoene and phytofluene are rarities among carotenoids due to their lower number of conjugated double bonds, as a result of which they absorb maximally in the UV region, with phytoene absorbing maximally in the UVB region and phytofluene in the UVA region. The compounds, which may be ingested or topically applied, are of great interest in the nutricosmetic field for their skin health and aesthetic benefits. Meléndez-Martinez et al 2018 (Journal of Food Composition and Analysis, 67, 91-103) discusses health and cosmetic benefits of phytoene and phytofluene. A review by Meléndez-Martinez et al 2015 (Archives of Biochemistry and Biophysics, 2015, 572, 188-200) discussed the possible beneficial effect of phytoene and phytofluene, concluding that these compounds may provide antioxidant activity, anticarcinogenic activity, anti-inflammatory activity, or protection against UVR-induced damage.

Chemical Structures of (A) Phytoene and (B) Phytofluene:

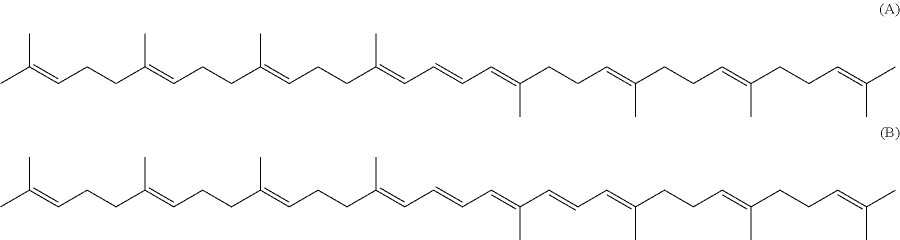

Ben-Amotz et al (J Phycol (2987) 23:176-181) reported an increase in the phytoene content, and corresponding decrease in the β-carotene content, of Dunaliella bardawil treated with the herbicide norflurazon, a phytoene desaturase inhibitor.

Dunaliella algae are also known to produce significant concentrations of α-carotene (IUPAC name 1,3,3-trimethyl-2-[(1E,3E,5E,7E,9E,11E,13E,15E,17E)-3,7,12,16-tetramethyl-18-(2,6,6-trimethylcyclohex-2-en-1-yl)octadeca-1,3,5,7,9,11,13,15,17-nonaenyl]cyclohexene): α-carotene has proven anti-metastatic action, which is not associated with provitamin A activity (Liu et al.; J Nutr Biochem. 2015 June; 26(6):607-15). The structure of α-carotene is shown below:

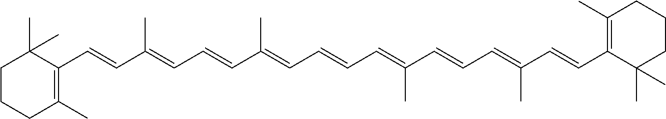 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

### SUMMARY OF THE INVENTION

The invention provides a Dunaliella alga, or extract thereof, comprising
i. an increased 9-cis β-carotene content and/or
ii. an increased colourless carotenoid content; and/or
iii. an increased α-carotene content;

when compared to a Dunaliella alga, or extract thereof, which is grown or cultivated under natural light or white light conditions.

The invention further provides a powdered Dunaliella alga, or extract thereof, comprising:
i. an increased 9-cis β-carotene content and/or
ii. an increased colourless carotenoid content; and/or
iii. an increased α-carotene content;

when compared to a Dunaliella alga, or extract thereof, which is grown or cultivated under natural light or white light conditions.

The invention further provides Dunaliella alga, or extract thereof; or a powdered Dunaliella alga, or extract thereof; comprising a 9-cis β-carotene content of 60 wt % of total carotenoids or greater.

The invention further provides a Dunaliella alga, or extract thereof; or a powdered Dunaliella alga, or extract thereof; comprising a colourless carotenoid content of 10 wt % of total carotenoids or greater.

The invention further provides a process for the preparation of a Dunaliella alga comprising exposing the Dunaliella alga to light of wavelength 500-1000 nm; and/or eliminating light of wavelength less than 500 nm (blue light).

The invention further provides the use of a Dunaliella alga or extract thereof, or a powdered Dunaliella alga or extract thereof, as a food colourant or food ingredient; or as a health supplement; or in a cosmetic composition, wherein the Dunaliella alga, or extract thereof, comprises
i. an increased 9-cis β-carotene content and/or
ii. an increased colourless carotenoid content; and/or
iii. an increased α-carotene content;

when compared to a Dunaliella alga, or extract thereof, which is grown or cultivated under natural light or white light conditions.

The invention further provides a Dunaliella alga or extract thereof, or a powdered Dunaliella alga or extract thereof, for use in therapy, wherein the Dunaliella alga, or extract thereof, comprises
i. an increased 9-cis β-carotene content and/or
ii. an increased colourless carotenoid content; and/or
iii. an increased α-carotene content;

when compared to a Dunaliella alga, or extract thereof, which is grown or cultivated under natural light or white light conditions.

The invention further provides a composition comprising: a) a Dunaliella alga, or extract thereof; or a powdered Dunaliella alga, or extract thereof and b) a pharmaceutically acceptable excipient, wherein the Dunaliella alga, or extract thereof, comprises
i. an increased 9-cis β-carotene content and/or
ii. an increased colourless carotenoid content; and/or
iii. an increased α-carotene content;

when compared to a Dunaliella alga, or extract thereof, which is grown or cultivated under natural light or white light conditions.

The invention further provides a process for the preparation of a Dunaliella alga comprising treating the Dunaliella alga by application of a herbicide selected from the group consisting of amino acid synthesis inhibitors, growth regulators, nitrogen metabolism inhibitor, pigment inhibitors, seedling root growth inhibitors, seedling shoot growth inhibitors, cell wall synthesis inhibitors, mitosis microtubule organisation inhibitors, and combinations thereof.

### BRIEF DESCRIPTION OF THE DRAWINGS

FIGS. 1A-1C show HPLC profiles at 450 nm of carotenoid extracts from Dunaliella salina exposed to continuous (FIG. 1A) white light, (FIG. 1B) red light and (FIG. 1C) blue light, each at 1000 μmol m⁻²s⁻¹ for 48 hours.

FIGS. 2A-2C show the effect of different light treatments on (FIG. 2A) the ratio of 9-cis and all-trans β-carotene, (FIG. 2B) the cellular content of 9-cis β-carotene and all-trans β-carotene, (FIG. 2C) the amount of 9-cis β-carotene as a % of the total amount of carotenoid in Dunaliella salina when cultivated to early orange phase until light treatment (T0) and then subjected to different light treatments for 48 hours.

FIGS. 3A-3B show the effect of different light treatments on (FIG. 3A) the cellular content of total carotenoids and chlorophyll, and (FIG. 3B) the cellular content of phytoene and all-trans α-carotene in Dunaliella salina when cultivated to early orange phase until light treatment (T0) and then subjected to different light treatments for 48 hours.

FIGS. 4A-4B show the effect of different light treatments on (FIG. 4A) the cellular content of 9-cis β-carotene and all-trans β-carotene, and (FIG. 4B) the ratio of 9-cis and all-trans β-carotene in Dunaliella salina when cultivated to mid-log phase (green phase) of growth until light treatment (T0) and then subjected to different light treatments.

FIGS. 5A-5C show the effect of different light treatments on (FIG. 5A) the cellular content of chlorophyll and total carotenoids, (FIG. 5B) the ratio of total carotenoids to total chlorophyll and (FIG. 5C) the cellular content of phytoene and all-trans α-carotene in Dunaliella salina when cultivated to mid-log phase of growth until light treatment (T0) and then subjected to different light treatments.

FIGS. 6A-6E show the cellular content of (FIG. 6A) 9-cis β-carotene, and of (FIG. 6B) all-trans β-carotene, (FIG. 6C) the ratio of 9-cis and all-trans β-carotene, (FIG. 6D) the cellular content of phytoene and (FIG. 6E) the cellular content of all-trans α-carotene in Dunaliella salina treated with either continuous blue or red LED light at three different light intensities.

FIGS. 7A-7C show the cellular content of (FIG. 7A) total carotenoids and (FIG. 7B) chlorophyll, and (FIG. 7C) the ratio of total carotenoids to total chlorophyll in Dunaliella salina treated with either continuous blue or red LED light at three different light intensities.

FIGS. 8A-8B show the effect of temperature on the cellular content of 9-cis β-carotene and all-trans β-carotene (FIG. 8A) and the ratios of 9-cis and all-trans β-carotene (FIG. 8B) in Dunaliella salina cells exposed to red or blue LED light.

FIGS. 9A-9F show the light properties of typical filters that may be used to transmit red light, such as: (purchased from Lee Filters) (FIG. 9A) 26 Bright red, (FIG. 9B) 27 Medium Red, and (FIG. 9C) 787 Marius Red; and of typical filters that eliminate blue light, such as: (purchased from Lee Filters), (FIG. 9D) 105 Orange and (FIG. 9E) 010 Medium Yellow. FIG. 9F shows the typical relative spectral power distribution of white, blue and red LED lights.

FIGS. 10A-10C show the effects of exposure of all-trans 1-carotene to red light under nitrogen (FIG. 10A) or in air (FIG. 10B) or to blue light under air or nitrogen (FIG. 10C).

FIG. 11 shows the classification of Dunaliella strains.

FIGS. 12A-12D show the effect of different white, dark and red light cycles applied to D. salina cultures over 72 h on the production of 9-cis- and all-trans β-carotenes and total carotenoids. Compensation for the intensity of light emitted by LED lights may be required when red filters are applied as covers to LED lights.

FIGS. 13A-13E show the effect of red light, far-red light of 730 nm, and light of 830 nm applied to D. salina cultures for 48 h on the ratio of 9-cis: all-trans β-carotene. Both far red light and red light increase the 9-cis/all-trans ratio compared to white light alone.

FIGS. 14A-14E show the effect of cultivating D. salina under different red/dark cycles of increasing red light cycle time on cell density (FIG. 14A), cellular content of total carotenoids (FIG. 14B), ratio of carotenoids:chlorophyll (FIG. 14C), cellular content of 9-cis β-carotene (FIG. 14D) and 9-cis: all-trans β-carotene ratio (FIG. 14E). The data show that continuous red light applied over 140 h reduces chlorophyll content but increases cell density, and total carotenoid content especially 9-cis β-carotene content.

FIGS. 15A-15E show the effect of treating D. salina cultures at 25° C. under either white LED light or red LED light in the presence of a phytoene desaturase inhibitor such as norflurazon.

FIGS. 16A-16J show the effect of cultivation of D. salina in the presence of chlorpropham.

FIGS. 17A-17C show the effect of cultivation of D. salina in the presence of the herbicides aminopyralid, carbetamide, and chlorsulfuron (cell division inhibitors), and glyphosate (phytochrome inhibitor).

FIGS. 18A-18J provide data to substantiate the identity of phytoene and phytofluene in cultures of D. salina.

FIG. 19 illustrates the carotenoid biosynthetic pathway.

### DETAILED DESCRIPTION

The inventors have surprisingly found that when exposed to red light (light of wavelength approximately 500 to 700 nm), eliminating blue light (light of wavelength less than 500 nm), green Dunaliella alga produces an increased content of all carotenoids, including phytoene, α-carotene and β-carotene, compared with the content produced by Dunaliella algae cultivated under normal white light (for example natural sun light). Alternatively, the Dunaliella alga may be exposed to red light of approximately 500 nm-700 nm and/or far-red light, and/or infrared light of wavelength approximately 700-1000 nm, preferably of wavelength approximately 500 nm to less than 830 nm. In particular, the ratio of 9-cis: all-trans-β-carotene is increased, therefore providing an improved yield of β-carotene product which has the additional advantage of being easier to formulate and administer due to the higher 9-cis: all-trans-β-carotene ratio. The relative increase in ratio of 9-cis: all-trans β-carotene on exposure to red light compared to white light is even greater using early-orange phase algae and even greater still when Dunaliella algae are cultivated during red light exposure under cool temperatures (for example 15° C. compared to 25° C.). Light filters that blocked out blue light wavelengths (400 nm-500 nm) from white light were also found to be effective in increasing the amount of 9-cis β-carotene and the ratio of 9-cis: all-trans β-carotene. In contrast, exposure to blue light decreased the amount of 9-cis β-carotene and the ratio of 9-cis: all-trans β-carotene produced by the Dunaliella alga. Furthermore, when cultivated under natural light, the properties of the Dunaliella alga vary seasonally, for example in content of carotenoids and/or colour. Such seasonal variation is reduced or eliminated when the Dunaliella alga is exposed to red light.

In embodiment 1, the invention provides a Dunaliella alga, or extract thereof, comprising
i. an increased 9-cis β-carotene content and/or
ii. an increased colourless carotenoid content; and/or
iii. an increased α-carotene content;

when compared to a Dunaliella alga, or extract thereof, which is grown or cultivated under natural light or white light conditions.

In embodiment 2, the invention provides a powdered Dunaliella alga, or extract thereof, comprising:
i. an increased 9-cis β-carotene content and/or
ii. an increased colourless carotenoid content; and/or
iii. an increased α-carotene content;

when compared to a Dunaliella alga, or extract thereof, which is grown or cultivated under natural light or white light conditions.

In embodiment 3, the invention provides a Dunaliella alga, or extract thereof; or a powdered Dunaliella alga, or extract thereof; comprising a 9-cis β-carotene content of 60 wt % of total carotenoids or greater.

In embodiment 4, the invention provides a Dunaliella alga, or extract thereof according to embodiment 1; or a powdered Dunaliella alga, or extract thereof according to embodiment 2; wherein the 9-cis β-carotene content is 60 wt % of total carotenoids or greater.

In embodiment 5, the invention provides a Dunaliella alga, or extract thereof; or a powdered Dunaliella alga, or extract thereof; according to any preceding embodiment, wherein the 9-cis β-carotene content is 65 wt % of total carotenoids or greater.

In embodiment 6, the invention provides a Dunaliella alga, or extract thereof; or a powdered Dunaliella alga, or extract thereof; according to any preceding embodiment, wherein the 9-cis β-carotene content is 70 wt % of total carotenoids or greater

In embodiment 7, the invention provides a Dunaliella alga, or extract thereof; or a powdered Dunaliella alga, or extract thereof; according to any preceding embodiment, wherein the 9-cis β-carotene content is 75 wt % of total carotenoids or greater.

In embodiment 8, the invention provides a Dunaliella alga, or extract thereof; or a powdered Dunaliella alga, or extract thereof; according to any preceding embodiment, wherein the β-carotene has a 9-cis: all-trans ratio of 1.2 or greater.

In embodiment 9, the invention provides a Dunaliella alga, or extract thereof; or a powdered Dunaliella alga, or extract thereof; according to any preceding embodiment, wherein the β-carotene has a 9-cis: all-trans ratio of 1.5 or greater.

In embodiment 10, the invention provides a Dunaliella alga, or extract thereof; or a powdered Dunaliella alga, or extract thereof; according to any preceding embodiment, wherein the β-carotene has a 9-cis: all-trans ratio 2.0 or greater.

In embodiment 11, the invention provides a Dunaliella alga, or extract thereof; or a powdered Dunaliella alga, or extract thereof; according to any preceding embodiment, wherein the β-carotene has a 9-cis: all-trans ratio 3.0 or greater.

In embodiment 12, the invention provides a Dunaliella alga, or extract thereof; or a powdered Dunaliella alga, or extract thereof; comprising a colourless carotenoid content of 10 wt % of total carotenoids or greater.

In embodiment 13, the invention a Dunaliella alga, or extract thereof; or a powdered Dunaliella alga, or extract thereof; according to any preceding embodiment, wherein the colourless carotenoid content is 11 wt % or greater.

In embodiment 14, the invention a Dunaliella alga, or extract thereof; or a powdered Dunaliella alga, or extract thereof; according to any preceding embodiment, wherein the colourless carotenoid content is 12 wt % or greater.

In embodiment 15, the invention provides a Dunaliella alga, or extract thereof; or a powdered Dunaliella alga, or extract thereof; according to any preceding embodiment, wherein the 9-cis β-carotene content is 60 wt % of total carotenoids or greater and the colourless carotenoid content is 10 wt % or greater of total carotenoids.

In embodiment 16, the invention provides a Dunaliella alga, or extract thereof; or a powdered Dunaliella alga, or extract thereof; according to any preceding embodiment, wherein the 9-cis β-carotene content is 60 wt % of total carotenoids or greater and the colourless carotenoid content is 11 wt % or greater of total carotenoids.

In embodiment 17, the invention provides a Dunaliella alga, or extract thereof; or a powdered Dunaliella alga, or extract thereof; wherein the 9-cis β-carotene content is 30 wt % of total carotenoids or greater and the colourless carotenoid content is 40 wt % or greater of total carotenoids.

In embodiment 18, the invention provides a Dunaliella alga, or extract thereof; or a powdered Dunaliella alga, or extract thereof; wherein the 9-cis β-carotene content is 60 wt % of total carotenoids or greater and the colourless carotenoid content is 4 wt % or greater of total carotenoids.

In embodiment 19, the invention provides a Dunaliella alga, or extract thereof; or a powdered Dunaliella alga, or extract thereof; wherein the 9-cis β-carotene content is 35 wt % of total carotenoids or greater and the colourless carotenoid content is 45 wt % or greater of total carotenoids.

In embodiment 20, the invention provides a Dunaliella alga, or extract thereof; or a powdered Dunaliella alga, or extract thereof; according to any preceding embodiment, wherein the colourless carotenoid content is the combined content of phytoene and phytofluene.

In embodiment 21, the invention provides a Dunaliella alga, or extract thereof; or a powdered Dunaliella alga, or extract thereof; according to any preceding embodiment, comprising a phytoene content of 10 wt % of total carotenoids or greater.

In embodiment 22, the invention provides a Dunaliella alga, or extract thereof; or a powdered Dunaliella alga, or extract thereof; according to any preceding embodiment, comprising a phytoene content of 11 wt % of total carotenoids or greater.

In embodiment 23, the invention provides a Dunaliella alga, or extract thereof; or a powdered Dunaliella alga, or extract thereof; according to any preceding embodiment, comprising a phytoene content of 12 wt % of total carotenoids or greater.

In embodiment 24, the invention provides a Dunaliella alga, or extract thereof; or a powdered Dunaliella alga, or extract thereof; optionally according to any preceding embodiment, comprising a phytoene content of 15 wt % of total carotenoids or greater.

In embodiment 25, the invention provides a Dunaliella alga, or extract thereof; or a powdered Dunaliella alga, or extract thereof; optionally according to any preceding embodiment, comprising a phytoene content of 20 wt % of total carotenoids or greater.

In embodiment 26, the invention provides a Dunaliella alga, or extract thereof; or a powdered Dunaliella alga, or extract thereof; optionally according to any preceding embodiment, comprising a phytoene content of 25 wt % of total carotenoids or greater.

In embodiment 27, the invention provides a Dunaliella alga, or extract thereof; or a powdered Dunaliella alga, or extract thereof; optionally according to any preceding embodiment, comprising a phytoene content of 30 wt % of total carotenoids or greater.

In embodiment 28, the invention provides a Dunaliella alga, or extract thereof; or a powdered Dunaliella alga, or extract thereof; optionally according to any preceding embodiment; comprising a phytoene content of 40 wt % of total carotenoids or greater.

In embodiment 29, the invention provides a Dunaliella alga, or extract thereof; or a powdered Dunaliella alga, or extract thereof; according to any preceding embodiment, comprising a phytoene content of 45 wt % of total carotenoids or greater.

For the avoidance of doubt, the content of total carotenoids will always total 100 wt %. In embodiment 30, the invention provides a Dunaliella alga, or extract thereof; or a powdered Dunaliella alga, or extract thereof; according to any preceding embodiment, wherein the Dunaliella alga is selected from Dunaliella salina salina, Dunaliella salina bardawil and Dunaliella salina rubeus (accession number CCAP 19/41).

In embodiment 31, the invention provides a composition comprising: a) a Dunaliella alga, or extract thereof; or a powdered Dunaliella alga, or extract thereof; according to any preceding embodiment; and b) a pharmaceutically acceptable excipient.

In embodiment 32, the invention provides a process for the preparation of a Dunaliella alga comprising exposing the Dunaliella alga to light of wavelength 500-1000 nm or 500-700 nm or 700-1000 nm; and/or eliminating light of wavelength less than 500 nm (blue light). The process preferably produces a Dunaliella alga which has increased 9-cis p-carotene content; and/or an increased colourless carotenoid content, particularly an increased phytoene content; and/or an increased α-carotene content.

In embodiment 33, the invention provides a process for the preparation of a Dunaliella alga comprising the steps:
a) cultivating the Dunaliella alga under white light; and subsequently;
b) exposing the Dunaliella alga to light of wavelength 500-1000 nm, or 500-700 nm or 700-1000 nm; and/or eliminating light of wavelength less than 500 nm (blue light).

In embodiment 34, the invention provides a process according to embodiment 32 or 33, wherein the step of exposing the Dunaliella alga to light of wavelength 500-1000 nm or 500-700 nm or 700-1000 nm; and/or eliminating light of wavelengths less than 500 nm (blue light); has a duration sufficient to achieve an increase in the 9-cis: all trans ration of 20% or greater; preferably 100% or greater; more preferably 150% or greater.

In embodiment 35, the invention provides a process according to embodiments 32 to 34, wherein the step of exposing the Dunaliella alga to light of wavelength 500-1000 nm or 500-700 nm or 700-1000 nm, preferably comprises the use of light of wavelength from greater than or equal to 500 to less than 830 nm.

In embodiment 36, the invention provides a process according to embodiments 32 to 35, wherein the step of exposing the Dunaliella alga to light of wavelength 500-1000 nm or 500-700 nm or 700-1000 nm, preferably comprises the use of light of wavelength 550-800 nm.

In embodiment 37, the invention provides a process according to embodiments 32 to 36, wherein the step of exposing the Dunaliella alga to light of wavelength 500-1000 nm or 500-700 nm or 700-1000 nm, preferably comprises the use of light of wavelength 600-750 nm.

In embodiment 38, the invention provides a process according to embodiments 32 to 37, wherein the step of exposing the Dunaliella alga to light of wavelength 500-1000 nm or 500-700 nm or 700-1000 nm, preferably comprises the use of light of wavelength 650-750 nm.

In embodiment 39, the invention provides a process according to embodiments 32 to 36, wherein the step of exposing the Dunaliella alga to light of wavelength 500-1000 nm or 500-700 nm or 700-1000 nm, preferably comprises the use of light of wavelength 600-700 nm or 650-700 nm.

The process according to embodiments 32 to 39 may be used for the cultivation of any strains of Dunaliella that produce carotenoids; preferably the Dunaliella alga is selected from Dunaliella salina salina, Dunaliella salina bardawil and Dunaliella salina rubeus (accession number CCAP 19/41).

In embodiment 40, the invention provides a process according to any one of embodiments 32 to 39, wherein the step of exposing the Dunaliella alga to light of wavelength 500-1000 nm, or 500-700 nm or 700-1000 nm, preferably from greater than or equal to 500 to less than 830 nm, preferably 550-800 nm, more preferably 600-750 nm, more preferably 650-750 nm, and more preferably 600-700 or 650-700 nm; and/or eliminating light of wavelength less than 500 nm (blue light); has a duration at least 4 hours.

In embodiment 41, the invention provides a process according to any one of embodiments 32 to 40, wherein the step of exposing the Dunaliella alga to light of wavelength 500-1000 nm, or 500-700 nm or 700-1000 nm, preferably from greater than or equal to 500 to less than 830 nm, preferably 550-800 nm, more preferably 600-750 nm, more preferably 650-750 nm, and more preferably 600-700 or 650-700 nm; and/or eliminating light of wavelength less than 500 nm (blue light); has a duration at least 12 hours.

In embodiment 42, the invention provides a process according to any one of embodiments 32 to 41, wherein the step of exposing the Dunaliella alga to light of wavelength 500-1000 nm, or 500-700 nm or 700-1000 nm, preferably from greater than or equal to 500 to less than 830 nm, preferably 550-800 nm, more preferably 600-750 nm, more preferably 650-750 nm, and more preferably 600-700 or 650-700 nm; and/or eliminating light of wavelength less than 500 nm (blue light); has a duration at least 24 hours.

In embodiment 43, the invention provides a process according to any one of embodiments 32 to 42, wherein the step of exposing the Dunaliella alga to light of wavelength 500-1000 nm, or 500-700 nm or 700-1000 nm, preferably from greater than or equal to 500 to less than 830 nm, preferably 550-800 nm, more preferably 600-750 nm, more preferably 650-750 nm, and more preferably 600-700 or 650-700 nm; and/or eliminating light of wavelength less than 500 nm (blue light); has a duration at least 48 hours.

The cultivation step a) of embodiments 33 to 43 may comprise cultivating the Dunaliella alga using any suitable method, such as in open pond systems, including cascade raceways and conventional raceways; and in closed cultivation systems, including tubular, flat-panel, green wall and thin-layer photobioreactors (PBRs). The cultivation step a) of embodiments 33 to 43 may take place outdoors or indoors, including in greenhouses.

The white light used in step a) of embodiments 33 to 43 may be any suitable source of white light, including natural light and white LED light.

The light used in step b) of embodiments 32 to 43 may be any suitable source of light of the desired wavelength, such as use of a red LED light, far-red light, or infrared light; or the use of a red filter such as the commercially available filters 26 Bright red, 27 Medium Red and 787 Marius Red (available from LEE Filters); or use of a filter that eliminates blue light, such as the commercially available filters 105 Orange, 101 Yellow, or 010 Medium Yellow. Far red light sources are known in the horticultural field.

In embodiment 44, the invention provides a process according to any one of embodiments 33 to 43, wherein step a) comprises cultivating the Dunaliella alga under natural light for a period from the beginning of cultivation to at least the log growth phase; preferably to the early orange phase.

In embodiment 45, the invention provides a process according to any one of embodiments 33 to 44, wherein in step b) the light has a wavelength in the range of from 650 nm to 700 nm and has an intensity of at least 10 μmol m⁻²s⁻¹.

In embodiment 46, the invention provides a process according to any one of embodiments 33 to 45 wherein in step b) the light of the desired wavelength is applied using a red filter; or using a red LED light, far-red light or infrared light; or using an orange or yellow filter which eliminates light of wavelength less than 500 nm.

In embodiment 47, the invention provides a process according to any one of embodiments 32 to 46, wherein the step of exposing the Dunaliella alga to light of wavelength 500-1000 nm is carried out at a temperature of 20° C. or less.

In embodiment 48, the invention provides a process according to any one of embodiments 32 to 47, wherein the step of exposing the Dunaliella alga to light of wavelength 500-1000 nm is carried out at a temperature of 15° C. or less.

In embodiment 49, the invention provides a process according to any one of embodiments 32 to 48, wherein the step of exposing the Dunaliella alga to light of wavelength 500-1000 nm is carried out at a temperature of 12° C. or less.

In embodiment 50, the invention provides a process according to any one of embodiments 33 to 49, which comprises the additional steps:
c) Harvesting the Dunaliella alga; and optionally
d) Extracting the carotenoids.

In step c) of embodiment 50, the Dunaliella alga may be harvested by any suitable method; preferably using a centrifuge or membrane micro/ultrafiltration.

In step d) of embodiment 50, the carotenoids may be extracted by any suitable process known to a person skilled in the art, such as extraction into a suitable organic solvent, or using supercritical CO₂, or any of the methods described in MAki-Arvela, et al (J. Chem. Technol. Biotechnol., 2014; 89: 1607-1626) or in Saini et al (Food Chemistry, 2018, 240, 90-103).

In embodiment 51, the invention provides a process according to any one of embodiments 31 to 50, wherein the Dunaliella alga is selected from any Dunaliella strain that produces carotenoids; preferably the Dunaliella alga is selected from Dunaliella salina salina, Dunaliella salina bardawil and Dunaliella salina rubeus (accession number CCAP 19/41).

In embodiment 52, the invention provides a process according to any one of embodiments 32 to 51, wherein in step a) the ambient temperature is in the range of from 4° C. to 45° C. The skilled person will understand that the temperature may vary during the cultivation step a) within the range of summer day time temperatures of up to 45° C. and winter night time temperatures down to 4° C.

The inventors have further surprisingly found that production of the colourless carotenoids phytoene and phytofluene by a Dunaliella alga or extract thereof is increased through the application of a herbicide. Thus, in embodiment 53, the invention provides a process according to any one of embodiments 32 to 52, which process comprises the steps:
a) cultivating the Dunaliella alga under white light; subsequently;
b) exposing the Dunaliella alga to light of wavelength 500-1000 nm, or 500-700 nm or 700-1000 nm; and/or eliminating light of wavelength less than 500 nm (blue light); and
c) applying a herbicide to the Dunaliella alga during step a) and/or step b).

For the avoidance of doubt, the term ‘a herbicide’ as used herein refers to a singular herbicide and to combinations of herbicides. When combinations of herbicides are used in the present invention, the herbicides may be applied simultaneously or sequentially.

Phytoene desaturase inhibitors, such as norflurazon, diflufenican and picolinafen, are known to have an effect on the accumulation of phytoene in Dunaliella alga. By inhibiting the activity of the phytoene desaturase (PDS), the carotenoid pathway is interrupted and the transformation of phytoene into other carotenoids is reduced. The inventors have now surprisingly found that phytoene and phytofluene content in Dunaliella alga can also be increased by treating the Dunaliella alga by application of a herbicide which is a cell division and phytochrome inhibitor, such as Chlorpropham, and postulate that such herbicides act by modulation of phytoene synthase, that is, by increasing the production of phytoene and phytofluene in the carotenoid pathway rather than by reducing the transformation of phytoene as has been seen with the application of a PDS inhibitor herbicide.

In addition to phytoene desaturase inhibitors, suitable herbicides for use in the present invention include those listed in the table below.

| Mode of | | |
|---|---|---|
| action (effect | Site of action | |
| on plant | and WSSA | Active ingredient |
| growth) | group* | (IUPAC name; CAS number) |
| AMINO ACID | ALS | Amidosulfuron |
| SYNTHESIS | INHIBITORS | (1-(4,6-dimethoxypyrimidin-2-yl)-3- |
| INHIBITORS | (acetolactate | [methyl(methylsulfonyl)sulfamoyl]urea; 120923-37-7 |
| | synthase) | Azimsulfuron |
| | Group 2 | 1-(4,6-dimethoxypyrimidin-2-yl)-3-[2-methyl-4-(2- |
| | | methyltetrazol-5-yl)pyrazol-3-yl]sulfonylurea; 120162- |
| | | 55-2 |
| | | bensulfuron-methyl |
| | | methyl 2-[(4,6-dimethoxypyrimidin-2- |
| | | yl)carbamoylsulfamoylmethyl]benzoate; 83055-99-6 |
| | | chlorimuron-ethyl |
| | | ethyl 2-[(4-chloro-6-methoxypyrimidin-2- |
| | | yl)carbamoylsulfamoyl]benzoate; 90982-32-4 |
| | | chlorsulfuron |
| | | 1-(2-chlorophenyl)sulfonyl-3-(4-methoxy-6-methyl-1,3,5- |
| | | triazin-2-yl)urea; 64902-72-3 |
| | | cinosulfuron |
| | | 1-(4,6-dimethoxy-1,3,5-triazin-2-yl)-3-[2-(2- |
| | | methoxyethoxy)phenyl]sulfonylureacyclosulfamuron; |
| | | 94593-91-6 |
| | | ethametsulfuron-methyl |
| | | methyl 2-[[4-ethoxy-6-(methylamino)-1,3,5-triazin-2- |
| | | yl]carbamoylsulfamoyl]benzoate; 97780-06-8 |
| | | ethoxysulfuron |
| | | (2-ethoxyphenyl) N-[(4,6-dimethoxypyrimidin-2- |
| | | yl)carbamoyl]sulfamate; 126801-58-9 |
| | | flazasulfuron |
| | | 1-(4,6-dimethoxypyrimidin-2-yl)-3-[3- |
| | | (trifluoromethyl)pyridin-2-yl]sulfonylurea; 104040-78-0 |
| | | flupyrsulfuron-methyl-sodium |
| | | sodium; (4,6-dimethoxypyrimidin-2-yl)-[[3- |
| | | methoxycarbonyl-6-(trifluoromethyl)pyridin-2- |
| | | yl]sulfonylcarbamoyl]azanide; 144740-54-5 |
| | | foramsulfuron |
| | | 2-[(4,6-dimethoxypyrimidin-2-yl)carbamoylsulfamoyl]-4- |
| | | formamido-N,N-dimethylbenzamide; 173159-57-4 |
| | | halosulfuron-methyl |
| | | methyl 3-chloro-5-[(4,6-dimethoxypyrimidin-2- |
| | | yl)carbamoylsulfamoyl]-1-methylpyrazole-4-carboxylate; |
| | | 100784-20-1 |
| | | imazosulfuron |
| | | 1-(2-chloroimidazo[1,2-a]pyridin-3-yl)sulfonyl-3-(4,6- |
| | | dimethoxypyrimidin-2-yl)urea; 122548-33-8 |
| | | iodosulfuron |
| | | 4-iodo-2-[(4-methoxy-6-methyl-1,3,5-triazin-2- |
| | | yl)carbamoylsulfamoyl]benzoic acid |
| | | mesosulfuron |
| | | 2-[(4,6-dimethoxypyrimidin-2-yl)carbamoylsulfamoyl]-4- |
| | | (methanesulfonamidomethyl)benzoic acid; 400852-66-6 |
| | | metsulfuron-methyl |
| | | methyl 2-[(4-methoxy-6-methyl-1,3,5-triazin-2- |
| | | yl)carbamoylsulfamoyl]benzoate; 74223-64-6 |
| | | nicosulfuron |
| | | 2-[(4,6-dimethoxypyrimidin-2-yl)carbamoylsulfamoyl]- |
| | | N,N-dimethylpyridine-3-carboxamide; 111991-09-4 |
| | | oxasulfuron |
| | | oxetan-3-yl 2-[(4,6-dimethylpyrimidin-2- |
| | | yl)carbamoylsulfamoyl]benzoate; 144651-06-9 |
| | | primisulfuron-methyl |
| | | methyl 2-[[4,6-bis(difluoromethoxy)pyrimidin-2- |
| | | yl]carbamoylsulfamoyl]benzoate; 86209-51-0 |
| | | prosulfuron |
| | | 1-(4-methoxy-6-methyl-1,3,5-triazin-2-yl)-3-[2-(3,3,3- |
| | | trifluoropropyl)phenyl]sulfonylurea; 94125-34-5 |
| | | pyrazosulfuron-ethyl |
| | | ethyl 5-[(4,6-dimethoxypyrimidin-2- |
| | | yl)carbamoylsulfamoyl]-1-methylpyrazole-4-carboxylate; |
| | | 93697-74-6 |
| | | rimsulfuron |
| | | 1-(4,6-dimethoxypyrimidin-2-yl)-3-(3- |
| | | ethylsulfonylpyridin-2-yl)sulfonylurea; 122931-48-0 |
| | | sulfometuron-methyl |
| | | methyl 2-[(4,6-dimethylpyrimidin-2- |
| | | yl)carbamoylsulfamoyl]benzoate; 74222-97-2 |
| | | sulfosulfuron |
| | | 1-(4,6-dimethoxypyrimidin-2-yl)-3-(2- |
| | | ethylsulfonylimidazo[1,2-a]pyridin-3-yl)sulfonylurea; |
| | | 141776-32-1 |
| | | thifensulfuron-methyl |
| | | methyl 3-[(4-methoxy-6-methyl-1,3,5-triazin-2- |
| | | yl)carbamoylsulfamoyl]thiophene-2-carboxylate; 79277- |
| | | 27-3 |
| | | triasulfuron |
| | | 1-[2-(2-chloroethoxy)phenyl]sulfonyl-3-(4-methoxy-6- |
| | | methyl-1,3,5-triazin-2-yl)urea; 82097-50-5 |
| | | tribenuron-methyl |
| | | methyl 2-[[(4-methoxy-6-methyl-1,3,5-triazin-2-yl)- |
| | | methylcarbamoyl]sulfamoyl]benzoate; 101200-48-0 |
| | | trifloxysulfuron |
| | | 1-(4,6-dimethoxypyrimidin-2-yl)-3-[3-(2,2,2- |
| | | trifluoroethoxy)pyridin-2-yl] sulfonylurea; 145099-21-4 |
| | | triflusulfuron-methyl |
| | | methyl 2-[[4-(dimethylamino)-6-(2,2,2-trifluoroethoxy)- |
| | | 1,3,5-triazin-2-yl]carbamoylsulfamoyl]-3-methylbenzoate; |
| | | 126535-15-7 |
| | | tritosulfuron |
| | | 1-[4-methoxy-6-(trifluoromethyl)-1,3,5-triazin-2-yl]-3-[2- |
| | | (trifluoromethyl)phenyl]sulfonylurea; 142469-14-5 |
| | | imazapic |
| | | 5-methyl-2-(4-methyl-5-oxo-4-propan-2-yl-1H-imidazol- |
| | | 2-yl)pyridine-3-carboxylic acid; 104098-48-8 |
| | | imazamethabenz-methyl |
| | | methyl 4-methyl-2-(4-methyl-5-oxo-4-propan-2-yl-1H- |
| | | imidazol-2-yl)benzoate; 69969-22-8 |
| | | imazamox |
| | | 5-(methoxymethyl)-2-(4-methyl-5-oxo-4-propan-2-yl-1H- |
| | | imidazol-2-yl)pyridine-3-carboxylic acid; 114311-32-9 |
| | | imazapyr |
| | | 2-(4-methyl-5-oxo-4-propan-2-yl-1H-imidazol-2- |
| | | yl)pyridine-3-carboxylic acid; 81334-34-1 |
| | | imazaquin |
| | | 2-(4-methyl-5-oxo-4-propan-2-yl-1H-imidazol-2- |
| | | yl)quinoline-3-carboxylic acid; 81335-46-8 |
| | | Imazethapyr |
| | | 5-ethyl-2-(4-methyl-5-oxo-4-propan-2-yl-1H-imidazol-2- |
| | | yl)pyridine-3-carboxylic acid; 81335-77-5 |
| | | cloransulam-methyl |
| | | methyl 3-chloro-2-[(5-ethoxy-7-fluoro-[1,2,4]triazolo[1,5- |
| | | c]pyrimidin-2-yl)sulfonylamino]benzoate; 147150-35-4 |
| | | diclosulam |
| | | N-(2,6-dichlorophenyl)-5-ethoxy-7-fluoro- |
| | | [1,2,4]triazolo[1,5-c]pyrimidine-2-sulfonamide; 145701- |
| | | 21-9 |
| | | florasulam |
| | | N-(2,6-difluorophenyl)-8-fluoro-5-methoxy- |
| | | [1,2,4]triazolo[1,5-c]pyrimidine-2-sulfonamide; 145701- |
| | | 23-1 |
| | | flumetsulam |
| | | N-(2,6-difluorophenyl)-5-methyl-[1,2,4]triazolo[1,5- |
| | | a]pyrimidine-2-sulfonamide; 98967-40-9 |
| | | metosulam |
| | | N-(2,6-dichloro-3-methylphenyl)-5,7-dimethoxy- |
| | | [1,2,4]triazolo[1,5-a]pyrimidine-2-sulfonamide; 139528- |
| | | 85-1 |
| | | penoxsulam |
| | | 2-(2,2-difluoroethoxy)-N-(5,8-dimethoxy- |
| | | [1,2,4]triazolo[1,5-c]pyrimidin-2-yl)-6- |
| | | (trifluoromethyl)benzenesulfonamide, 219714-96-2 |
| | | bispyribac-sodium |
| | | sodium; 2,6-bis[(4,6-dimethoxypyrimidin-2- |
| | | yl)oxy]benzoate; 125401-92-5 |
| | | pyribenzoxim |
| | | (benzhydrylideneamino) 2,6-bis[(4,6- |
| | | dimethoxypyrimidin-2-yl)oxy]benzoate; 168088-61-7 |
| | | pyriftalid |
| | | 7-(4,6-dimethoxypyrimidin-2-yl)sulfanyl-3-methyl-3H-2- |
| | | benzofuran-1-one; 135186-78-6 |
| | | pyrithiobac-sodium |
| | | sodium; 2-chloro-6-(4,6-dimethoxypyrimidin-2- |
| | | yl)sulfanylbenzoate; 123343-16-8 |
| | | pyriminobac-methyl |
| | | methyl 2-(4,6-dimethoxypyrimidin-2-yl)oxy-6-[(E)-N- |
| | | methoxy-C-methylcarbonimidoyl]benzoate; 136191-64-5 |
| | | flucarbazone-sodium |
| | | sodium; (3-methoxy-4-methyl-5-oxo-1,2,4-triazole-1- |
| | | carbonyl)-[2-(tnfluoromethoxy)phenyl]sulfonylazanide; |
| | | 181274-17-9 |
| | | propoxycarbazone-sodium |
| | | sodium; (2-methoxycarbonylphenyl)sulfonyl-(4-methyl-5- |
| | | oxo-3-propoxy-1,2,4-triazole-1-carbonyl)azanide; |
| | | 181274-15-7 |
| | EPSP | glyphosate |
| | SYNTHASE | 2-(phosphonomethylamino)acetic acid; 1071-83-6 |
| | INHIBITOR | sulfosate (glyphosate-trimesium) |
| | (5- | 2-(phosphonomethylamino)acetate; trimethylsulfanium; |
| | enolpyruvyl- | 81591-81-3 |
| | shikimate3- | |
| | phosphate) | |
| | Group 9 | |
| GROWTH | TIR1 AUXIN | Clomeprop |
| REGULATORS | RECEPTORS | 2-(2,4-dichloro-3-methylphenoxy)-N-phenylpropanamide; |
| | (synthetic | 84496-56-0 |
| | auxins) | 2,4-D |
| | Group 4 | 2-(2,4-dichlorophenoxy)acetic acid; 94-75-7 |
| | | 2,4-DB |
| | | 4-(2,4-dichlorophenoxy)butanoic acid; N- |
| | | methylmethanamine; 2758-42-1 |
| | | dichlorprop (2,4-DP) |
| | | 2-(2,4-dichlorophenoxy)propanoic acid; 120-36-5 |
| | | MCPA |
| | | 2-(4-chloro-2-methylphenoxy)acetic acid; 94-74-6 |
| | | MCPB |
| | | 4-(4-chloro-2-methylphenoxy)butanoic acid; 94-81-5 |
| | | mecoprop (MCPP or CMPP) |
| | | 2-(4-chloro-2-methylphenoxy)propanoic acid; 93-65-2 |
| | | chloramben |
| | | 3-amino-2,5-dichlorobenzoic acid; 133-90-4 |
| | | dicamba |
| | | 3,6-dichloro-2-methoxybenzoic acid; 1918-00-9 |
| | | thiobarbituric acid (TBA) |
| | | 2-sulfanylidene-1,3-diazinane-4,6-dione; 504-17-6 |
| | | clopyralid |
| | | 3,6-dichloropyridine-2-carboxylic acid; 1702-17-6 |
| | | fluroxypyr |
| | | 2-(4-amino-3,5-dichloro-6-fluoropyridin-2-yl)oxyacetic |
| | | acid; 69377-81-7 |
| | | picloram |
| | | 4-amino-3,5,6-trichloropyridine-2-carboxylic acid; 1918- |
| | | 02-1 |
| | | triclopyr |
| | | 2-(3,5,6-trichloropyridin-2-yl)oxyacetic acid; 55335-06-3 |
| | | quinclorac (also HRAC group L) |
| | | 3,7-dichloroquinoline-8-carboxylic acid; 84087-01-4 |
| | | quinmerac |
| | | 7-chloro-3-methylquinoline-8-carboxylic acid; 90717-03- |
| | | 6 |
| | | benazolin-ethyl |
| | | ethyl 2-(4-chloro-2-oxo-1,3-benzothiazol-3-yl)acetate; |
| | | 25059-80-7 |
| | AUXIN | naptalam |
| | TRANSPORT | 2-(naphthalen-1-ylcarbamoyl)benzoic acid; 132-66-1 |
| | INHIBITOR | diflufenzopyr-sodium |
| | Group 19 | sodium; 2-[(E)-N-[(3,5-difluorophenyl)carbamoylamino]- |
| | | C-methylcarbonimidoyl]pyridine-3-carboxylate; 109293- |
| | | 98-3 |
| NITROGEN | GLUTAMINE | glufosinate-ammonium |
| METABOLISM | SYNTHETASE | 2-amino-4-[hydroxy(methyl)phosphoryl]butanoic |
| INHIBITOR | INHIBITOR | acid; azane; 77182-82-2 |
| | Group 10 | bialaphos (bilanaphos) |
| | | (2S)-2-[[(2S)-2-[[(2S)-2-amino-4- |
| | | [hydroxy(methyl)phosphoryl]butanoyl]amino]pro- |
| | | panoyl]amino]propanoic acid; |
| PIGMENT | PHYTOENE | norflurazon |
| INHIBITORS | DESATURASE | 4-chloro-5-(methylamino)-2-[3- |
| | (PDS) | (trifluoromethyl)phenyl]pyridazin-3-one; 27314-13-2 |
| | INHIBITOR | diflufenican |
| | Group 12 | N-(2,4-difluorophenyl)-2-[3- |
| | | (trifluoromethyl)phenoxy]pyridine-3-carboxamide; |
| | | 83164-33-4 |
| | | picolinafen |
| | | N-(4-fluorophenyl)-6-[3- |
| | | (trifluoromethyl)phenoxy]pyridine-2-carboxamide; |
| | | 137641-05-5 |
| | | beflubutamid |
| | | N-benzyl-2-[4-fluoro-3- |
| | | (trifluoromethyl)phenoxy]butanamide; 113614-08-7 |
| | | fluridone |
| | | 1-methyl-3-phenyl-5-[3-(trifluoromethyl)phenyl]pyridin- |
| | | 4-one; 59756-60-4 |
| | | flurochloridone |
| | | 3-chloro-4-(chloromethyl)-1-[3- |
| | | (trifluoromethyl)phenyl]pyrrolidin-2-one; 61213-25-0 |
| | | flurtamone |
| | | 5-(methylamino)-2-phenyl-4-[3- |
| | | (trifluoromethyl)phenyl]furan-3-one; 96525-23-4 |
| | Bleaching | mesotrione |
| | HPPD | 2-(4-methylsulfonyl-2-nitrobenzoyl)cyclohexane-1,3- |
| | INHIBITORS | dione; 104206-82-8 |
| | Group 27 | sulcotrione |
| | | 2-(2-chloro-4-methylsulfonylbenzoyl)cyclohexane-1,3- |
| | | dione; 114680-61-4 |
| | | isoxachlortole |
| | | (4-chloro-2-methylsulfonylphenyl)-(5-cyclopropyl-1,2- |
| | | oxazol-4-yl)methanone; 141112-06-3 |
| | | isoxaflutole |
| | | (5-cyclopropyl-1,2-oxazol-4-yl)-[2-methylsulfonyl-4- |
| | | (trifluoromethyl)phenyl]methanone; 141112-29-0 |
| | | benzofenap |
| | | 2-[4-(2,4-dichloro-3-methylbenzoyl)-2,5-dimethylpyrazol- |
| | | 3-yl]oxy-1-(4-methylphenyl)ethenone; 82692-44-2 |
| | | pyrazolynate |
| | | [4-(2,4-dichlorobenzoyl)-2,5-dimethylpyrazol-3-yl] 4- |
| | | methylbenzenesulfonate; 58011-68-0 |
| | | pyrazoxyfen |
| | | 2-[4-(2,4-dichlorobenzoyl)-2,5-dimethylpyrazol-3-yl]oxy- |
| | | 1-phenylethanone; 71561-11-0 |
| | | benzobicyclon |
| | | 3-(2-chloro-4-methylsulfonylbenzoyl)-2- |
| | | phenylsulfanylbicyclo[3.2.1]oct-2-en-4-one; 156963-66-5 |
| | | bromobutide |
| | | 2-bromo-3,3-dimethyl-N-(2-phenylpropan-2- |
| | | yl)butanamide; 74712-19-9 |
| | | (chloro)-flurenol |
| | | 2-chloro-9-hydroxyfluorene-9-carboxylic acid; 2464-37-1 |
| | | cinmethylin |
| | | 1-methyl-2-[(2-methylphenyl)methoxy]-4-propan-2-yl-7- |
| | | oxabicyclo[2.2.1]heptane; 87818-31-3 |
| | | cumyluron |
| | | 1-[(2-chlorophenyl)methyl]-3-(2-phenylpropan-2-yl)urea; |
| | | 99485-76-4 |
| | | dazomet |
| | | 3,5-dimethyl-1,3,5-thiadiazinane-2-thione; 533-74-4 |
| | | dymron (daimuron) |
| | | 1-(4-methylphenyl)-3-(2-phenylpropan-2-yl)urea; 42609- |
| | | 52-9 |
| | | methyl-dymron (methyl-dimuron) |
| | | 1-methyl-1-phenyl-3-(2-phenylpropan-2-yl)urea; 42609- |
| | | 73-4 |
| | | etobenzanid |
| | | N-(2,3-dichlorophenyl)-4-(ethoxymethoxy)benzamide; |
| | | 79540-50-4 |
| | | fosamine |
| | | carbamoyl(ethoxy)phosphinic acid; 59682-52-9 |
| | | indanofan |
| | | 2-[[2-(3-chlorophenyl)oxiran-2-yl]methyl]-2-ethylindene- |
| | | 1,3-dione; 133220-30-1 |
| | | metam |
| | | methylcarbamodithioic acid; 144-54-7 |
| | | oxaziclomefone |
| | | 3-[2-(3,5-dichlorophenyl)propan-2-yl]-6-methyl-5- |
| | | phenyl-2H-1,3-oxazin-4-one; 153197-14-9 |
| | | oleic acid |
| | | (Z)-octadec-9-enoic acid; 112-80-1 |
| | | pelargonic acid |
| | | nonanoic acid; 112-05-0 |
| | | pyributicarb |
| | | O-(3-tert-butylphenyl) N-(6-methoxypyridin-2-yl)-N- |
| | | methylcarbamothioate; 88678-67-5 |
| | Inhibition of | amitrole |
| | carotenoid | (in vivo inhibition of |
| | biosynthesis | lycopene cyclase) |
| | (unknown | 1H-1,2,4-triazol-5-amine; 61-82-5 |
| | target) | |
| | Group 11 | |
| SEEDLING | MICROTUBULE | benefin (benfluralin) |
| ROOT | INHIBITORS | N-butyl-N-ethyl-2,6-dinitro-4-(trifluoromethyl)aniline; |
| GROWTH | Group 3 | 1861-40-1 |
| INHIBITORS | | butralin |
| | | N-butan-2-yl-4-tert-butyl-2,6-dinitroaniline; 33629-47-9 |
| | | dinitramine |
| | | 3-N,3-N-diethyl-2,4-dinitro-6-(trifluoromethyl)benzene- |
| | | 1,3-diamine; 29091-05-2 |
| | | ethalfluralin |
| | | N-ethyl-N-(2-methylprop-2-enyl)-2,6-dinitro-4- |
| | | (trifluoromethyl)aniline, 55283-68-6 |
| | | oryzalin |
| | | 4-(dipropylamino)-3,5-dinitrobenzenesulfonamide; |
| | | 19044-88-3 |
| | | pendimethalin |
| | | 3,4-dimethyl-2,6-dinitro-N-pentan-3-ylaniline; 40487-42- |
| | | 1 |
| | | trifluralin |
| | | 2,6-dinitro-N,N-dipropyl-4-(trifluoromethyl)aniline; |
| | | 1582-09-8 |
| | | amiprophos-methyl |
| | | N-[methoxy-(4-methyl-2- |
| | | nitrophenoxy)phosphinothioyl]propan-2-amine; 36001- |
| | | 88-4 |
| | | butamiphos |
| | | N-[ethoxy-(5-methyl-2- |
| | | nitrophenoxy)phosphinothioyl]butan-2-amine; 36335-67- |
| | | 8 |
| | | dithiopyr |
| | | 3-S,5-S-dimethyl 2-(difluoromethyl)-4-(2-methylpropyl)- |
| | | 6-(trifluoromethyl)pyridine-3,5-dicarbothioate; 97886-45- |
| | | 8 |
| | | thiazopyr |
| | | methyl 2-(difluoromethyl)-5-(4,5-dihydro-1,3-thiazol-2- |
| | | yl)-4-(2-methylpropyl)-6-(trifluoromethyl)pyridine-3- |
| | | carboxylate; 117718-60-2 |
| | | propyzamide (pronamide) |
| | | propenamide; 79-05-0 |
| | | tebutam |
| | | N-benzyl-2,2-dimethyl-N-propan-2-ylpropanamide; |
| | | 35256-85-0 |
| | | DCPA (chlorthal-dimethyl) |
| | | dimethyl 2,3,5,6-tetrachlorobenzene-1,4-dicarboxylate; |
| | | 1861-32-1 |
| SEEDLING | LONG- | acetochlor |
| SHOOT | CHAIN | 2-chloro-N-(ethoxymethyl)-N-(2-ethyl-6- |
| GROWTH | FATTY ACID | methylphenyl)acetamide; 34256-82-1 |
| INHIBITORS | INHIBITORS | alachlor |
| | (inhibition of | 2-chloro-N-(2,6-diethylphenyl)-N- |
| | cell division) | (methoxymethyl)acetamide; 15972-60-8 |
| | Group15 | butachlor |
| | | N-(butoxymethyl)-2-chloro-N-(2,6- |
| | | diethylphenyl)acetamide; 23184-66-9 |
| | | dimethachlor |
| | | 2-chloro-N-(2,6-dimethylphenyl)-N-(2- |
| | | methoxyethyl)acetamide; 50563-3 6-5 |
| | | dimethenamid |
| | | 2-Chloro-N-(2,4-dimethyl-3-thienyl)-N-(2-methoxy-1- |
| | | methylethyl)acetamide; |
| | | metazachlor |
| | | 2-chloro-N-(2,6-dimethylphenyl)-N-(pyrazol-1- |
| | | ylmethyl)acetamide; 67129-08-2 |
| | | metolachlor |
| | | 2-chloro-N-(2-ethyl-6-methylphenyl)-N-(1- |
| | | methoxypropan-2-yl)acetamide; 51218-45-2 |
| | | pethoxamid |
| | | 2-chloro-N-(2-ethoxyethyl)-N-(2-methyl-1-phenylprop-1- |
| | | enyl)acetamide; 106700-29-2 |
| | | pretilachlor |
| | | 2-chloro-N-(2,6-diethylphenyl)-N-(2- |
| | | propoxyethyl)acetamide; 51218-49-6 |
| | | propachlor |
| | | 2-chloro-N-phenyl-N-propan-2-ylacetamide; 1918-16-7 |
| | | propisochlor |
| | | 2-chloro-N-(2-ethyl-6-methylphenyl)-N-(propan-2- |
| | | yloxymethyl)acetamide; 86763-47-5 |
| | | thenylchlor |
| | | 2-chloro-N-(2,6-dimethylphenyl)-N-[(3- |
| | | methoxythiophen-2-yl)methyl]acetamide; 96491-05-3 |
| | | diphenamid |
| | | N,N-dimethyl-2,2-diphenylacetamide; 957-51-7 |
| | | napropamide |
| | | N,N-diethyl-2-naphthalen-1-yloxypropanamide; 15299- |
| | | 99-7 |
| | | naproanilide |
| | | 2-naphthalen-2-yloxy-N-phenylpropanamide; 52570-16-8 |
| | | flufenacet |
| | | N-(4-fluorophenyl)-N-propan-2-yl-2-[[5- |
| | | (trifluoromethyl)-1,3,4-thiadiazol-2-yl]oxy]acetamide; |
| | | 142459-58-3 |
| | | mefenacet |
| | | 2-(1,3-benzothiazol-2-yloxy)-N-methyl-N- |
| | | phenylacetamide; 73250-68-7 |
| | | fentrazamide |
| | | 4-(2-chlorophenyl)-N-cyclohexyl-N-ethyl-5-oxotetrazole- |
| | | 1-carboxamide; 158237-07-1 |
| | | anilofos |
| | | N-(4-chlorophenyl)-2-dimethoxyphosphinothioylsulfanyl- |
| | | N-propan-2-ylacetamide; 64249-01-0 |
| | | cafenstrole |
| | | N,N-diethyl-3-(2,4,6-trimethylphenyl)sulfonyl-1,2,4- |
| | | triazole-1-carboxamide; 125306-83-4 |
| | | piperophos |
| | | 2-dipropoxyphosphinothioylsulfanyl-1-(2- |
| | | methylpiperidin-1-yl)ethenone; 24151-93-7 |
| INHIBITION | INHIBITION | dichlobenil |
| OF CELL | OF CELL | 2,6-dichlorobenzonitrile; 1194-65-6 |
| WALL | WALL | chlorthiamide |
| SYNTHESIS | SYNTHESIS | 2,6-dichlorobenzenecarbothioamide; 1918-13-4 |
| | Group 20 | |
| INHIBITION | INHIBITION | chlorpropham |
| OF MITOSIS | OF MITOSIS | propan-2-yl N-(3-chlorophenyl)carbamate; 101-21-3 |
| MICROTUBULE | MICROTUBULE | propham |
| | | propan-2-yl N-phenylcarbamate; 122-42-9 |
| | | carbetamide |
| ORGANISATION | ORGANISATION | [(2R)-1-(ethylamino)-1-oxopropan-2-yl] N- |
| | Group 23 | phenylcarbamate; 16118-49-3 |
| UNKNOWN | Group 25 | arylaminopropionic acid |
| | | 3-(prop-2-enylamino)propanoic acid; |
| UNKNOWN | Group 26 | quinoline carboxylic acid |
| | | chlorocarbonic-acid |
| | | carbonochloridic acid; 463-73-0 |
| | | pyrazolium |
| | | 1H-pyrazol-2-ium; |
| UNKNOWN | Group 16 | benzofurane |

*site of action groups designated by the WSSA (Weed Science Society of America)

The active herbicidal ingredients listed above may be used as a free acid or base, or as a suitable salt. Where the compound possesses a chiral centre, the racemic form or a specific diastereoisomer or enantiomer may be used.

Particular suitable herbicides include:

Norflurazon [4-chloro-5-methylamino-2-(3-trifluoromethylphenyl)-pyridazin-3(2H)one] is a pyridazinone bleaching herbicide which inhibits carotene biosynthesis in photosynthetic organisms including D. salina, by binding reversibly in a non-competitive manner with its target enzyme phytoene desaturase. In Dunaliella sp it causes the accumulation of phytoene (Ben-Amotz A, Gressel J, Avron M (1987) Massive accumulation of phytoene induced by norflurazon in Dunaliella bardawil (Chlorophyceae) prevents recovery from photoinhibition. J Phycol 23:176-181), but not phytofluene (Ben-Amotz A, Lers A, Avron M (1988) Stereoisomers of beta carotene and phytoene in the alga Dunaliella bardawil. Plant Physiol 86:1286-1291). Other known phytoene desaturase (PDS) inhibitor herbicides, such as diflufenican and picolinafen, will also therefore permit phytoene accumulation and are suitable for use in the present invention.

Chlorpropham (isopropyl N-(3-chlorophenyl) carbamate (CIPC) (commercial names: Bud Nip, Taterpex, Preventol, Elbanil, Metoxon, Nexoval, Stickman Pistols, Preweed, Furloe, Stopgerme-S, Sprout Nip, Mirvale, Bygran, ChlorIPC, CHLOROPROPHAM, Spud-Nic, Spud-Nie, Chloro-IFK, Chloro-IPC, Keim-stop, Triherbicide CIPC) is a carbamate herbicide and plant growth regulator used for pre-emergence control of grass weeds in alfalfa, lima and snap beans, blueberries, cranberries, carrots, cranberries, ladino clover, garlic, seed grass, onions, spinach, sugar beets, tomatoes, safflower, soybeans, gladioli and woody nursery stock. In the post-harvest treatment of potatoes during storage and transport, it is also used as a sprout suppressant and for sucker control in tobacco. It is considered to be a phytochrome inhibitor (Mann et al 1967 Nature 213, 420-421), and in wheat, has been shown to disorganize cell microtubules and microtubule organizing centres to prevent cell division (Eleftheriou, E. & Bekiari, E. Plant and Soil (2000) 226: 11. Ultrastructural effects of the herbicide chlorpropham (CIPC) in root tip cells of wheat).

Aminopyralid (4-amino-3, 6-dichloropyridine-2-carboxylic acid) is a post-emergent, auxin-type herbicide that inhibits cell division and has been widely used for weed control. It is a member of the pyridine carboxylic acid family and induces an auxin-type response in susceptible plant species, causing epinastic bending and twisting of the stems that result in growth inhibition. (Li, W., et al (2018), Ecotoxicology and Environmental Safety, 155, 17-25).

Carbetamide ((R)-1-(ethylcarbamoyl)ethyl carbanilate) is a pre- and post-emergence herbicide which targets microtubule organizing centres and disrupts mitosis and cytokinesis in proliferating plant tissues, inhibiting cell division (Giménez-Abidn, M. I., Panzera, F., López-Sáez, J. F. et al. Protoplasma (1998) 204: 119).

Chlorsulfuron is a sulfonylurea herbicide which inhibits plant acetohydroxyacid synthase, the first enzyme in the branched-chain amino acid biosynthesis pathway and is closely associated with an inhibition of plant cell division.

Glyphosate acts as a transition state inhibitor of 5-enolpyruvylshikimate-3-phosphate synthase which is responsible for facilitating the assembly of shikimate-3-phosphate and phosphoenolpyruvate in the shikimate pathway and is a critical biosynthetic pathway in plant cellular plastids. (d'Avignon, Ge, (2018) J. Magnetic Resonance, 292, 59-72). It is also linked to phytochrome inhibition (Duke et al (1979), Effects of Glyphosate on Metabolism of Phenolic Compounds. Physiologia Plantarum, 46: 307-317).

In embodiment 54, the invention provides a process according to embodiment 53, wherein the herbicide is selected from amino acid synthesis inhibitors, growth regulators, nitrogen metabolism inhibitors, pigment inhibitors, seedling root growth inhibitors, seedling shoot growth inhibitors, cell wall synthesis inhibitors, mitosis microtubule organisation inhibitors, and combinations thereof.

In embodiment 55, the invention provides a process according to embodiment 53 or 54, wherein the herbicide is selected from acetolactate synthase (ALS) inhibitors, 5-enolpyruvyl-shikimate3-phosphate (EPSP) synthase inhibitors, transport inhibitor response (TIR) 1 auxin receptors (synthetic auxins), auxin transport inhibitors, glutamine synthetase inhibitors, phytoene desaturase inhibitors, bleaching 4-Hydroxyphenylpyruvate dioxygenase (HPPD) inhibitors, carotenoid biosynthesis inhibitors (unknown target), microtubule inhibitors, long-chain fatty acid inhibitors (cell division inhibitors), cell wall synthesis inhibitors, mitosis microtubule organization inhibitors, and combinations therefore.

In embodiment 56, the invention provides a process according to any one of embodiments 53 to 55, wherein the herbicide is selected from amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-sodium, foramsulfuron, halosulfuron-methyl, imazosulfuron, iodosulfuron, mesosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, penoxsulam, bispyribac-sodium, pyribenzoxim, pyriftalid, pyrithiobac-sodium, pyriminobac-methyl, flucarbazone-sodium, propoxycarbazone-sodium, glyphosate, sulfosate, clomeprop, 2,4-D, 2,4-DB, dichlorprop (2,4-DP), MCPA, MCPB, mecoprop (MCPP or CMPP), chloramben, dicamba, TBA, clopyralid, fluroxypyr, picloram, triclopyr, quinclorac, Quinmerac, benazolin-ethyl, naptalam, diflufenzopyr-sodium, glufosinate-ammonium, bialaphos (bilanaphos), Norflurazon, diflufenican, picolinafen, beflubutamid, fluridone, flurochloridone, flurtamone, mesotrione, sulcotrione, isoxachlortole, isoxaflutole, benzofenap, pyrazolynate, pyrazoxyfen, Benzobicyclon, bromobutide, (chloro)-flurenol, Cinmethylin, Cumyluron, Dazomet, dymron (daimuron), methyl-dymron (methyl-dimuron), etobenzanid, fosamine, indanofan, metam, oxaziclomefone, oleic acid, pelargonic acid, pyributicarb, amitrole, benefin (benfluralin), butralin, dinitramine, ethalfluralin, oryzalin, pendimethalin, trifluralin, amiprophos-methyl, butamiphos, dithiopyr, thiazopyr, propyzamide (pronamide), tebutam, propyzamide (pronamide), tebutam, chlorthal-dimethyl (DCPA), acetochlor, alachlor, butachlor, dimethachlor, dimethenamid, metazachlor, metolachlor, pethoxamid, pretilachlor, propachlor, propisochlor, thenylchlor, diphenamid, napropamide, naproanilide, flufenacet, mefenacet, fentrazamide, anilofos, cafenstrole, piperophos, dichlobenil, chlorthiamide, chlorpropham, propham, carbetamide, and combinations thereof.

In embodiment 57, the invention provides a process according to any one of embodiments 53 to 56, wherein the herbicide is selected from amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-sodium, foramsulfuron, halosulfuron-methyl, imazosulfuron, iodosulfuron, mesosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, penoxsulam, bispyribac-sodium, pyribenzoxim, pyriftalid, pyrithiobac-sodium, pyriminobac-methyl, flucarbazone-sodium, propoxycarbazone-sodium, glyphosate, sulfosate, benazolin-ethyl, glufosinate-ammonium, bialaphos (bilanaphos), norflurazon, diflufenican, picolinafen, beflubutamid, fluridone, flurochloridone, flurtamone, mesotrione, sulcotrione, isoxachlortole, isoxaflutole, benzofenap, pyrazolynate, pyrazoxyfen, Benzobicyclon, bromobutide, (chloro)-flurenol, Cinmethylin, Cumyluron, Dazomet, dymron (daimuron), methyl-dymron (methyl-dimuron), etobenzanid, fosamine, indanofan, metam, oxaziclomefone, oleic acid, pelargonic acid, pyributicarb, benefin (benfluralin), butralin, dinitramine, ethalfluralin, oryzalin, pendimethalin, trifluralin, amiprophos-methyl, butamiphos, dithiopyr, thiazopyr, propyzamide (pronamide), tebutam, propyzamide (pronamide), tebutam, chlorthal-dimethyl (DCPA), chlorpropham, propham, carbetamide, and combinations thereof.

In embodiment 58, the invention provides a process according to any one of embodiments 53 to 57, wherein the herbicide is selected from norflurazon, diflufenican, picolinafen, beflubutamid, fluridone, flurochloridone, flurtamone, chlorpropham, propham, carbetamide, and combinations thereof; most preferably chlorpropham.

In embodiment 59, the invention provides a process for the preparation of a Dunaliella alga, comprising treating the Dunaliella alga by applying a herbicide selected from the group consisting of amino acid synthesis inhibitors, growth regulators, nitrogen metabolism inhibitor, pigment inhibitors (excluding phytoene desaturase inhibitors), seedling root growth inhibitors, seedling shoot growth inhibitors, cell wall synthesis inhibitors, mitosis microtubule organisation inhibitors, and combinations thereof.

In embodiment 60, the invention provides a process according to embodiment 59, wherein the herbicide is selected from acetolactate synthase (ALS) inhibitors, 5-enolpyruvyl-shikimate3-phosphate (EPSP) synthase inhibitors, transport inhibitor response (TIR) 1 auxin receptors (synthetic auxins), auxin transport inhibitors, glutamine synthetase inhibitors, bleaching 4-Hydroxyphenylpyruvate dioxygenase (HPPD) inhibitors, carotenoid biosynthesis inhibitors (unknown target), microtubule inhibitors, long-chain fatty acid inhibitors (cell division inhibitors), cell wall synthesis inhibitors, mitosis microtubule organization inhibitors, and combinations therefore.

In embodiment 61, the invention provides a process according to embodiment 59 or 60, wherein the herbicide is selected from amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-sodium, foramsulfuron, halosulfuron-methyl, imazosulfuron, iodosulfuron, mesosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, penoxsulam, bispyribac-sodium, pyribenzoxim, pyriftalid, pyrithiobac-sodium, pyriminobac-methyl, flucarbazone-sodium, propoxycarbazone-sodium, glyphosate, sulfosate, clomeprop, 2,4-D, 2,4-DB, dichlorprop (2,4-DP), MCPA, MCPB, mecoprop (MCPP or CMPP), chloramben, dicamba, TBA, clopyralid, fluroxypyr, picloram, triclopyr, quinclorac, Quinmerac, benazolin-ethyl, naptalam, diflufenzopyr-sodium, glufosinate-ammonium, bialaphos (bilanaphos), mesotrione, sulcotrione, isoxachlortole, isoxaflutole, benzofenap, pyrazolynate, pyrazoxyfen, Benzobicyclon, bromobutide, (chloro)-flurenol, Cinmethylin, Cunyluron, Dazomet, dymron (daimuron), methyl-dymron (methyl-dimuron), etobenzanid, fosamine, indanofan, metam, oxaziclomefone, oleic acid, pelargonic acid, pyributicarb, amitrole, benefin (benfluralin), butralin, dinitramine, ethalfluralin, oryzalin, pendimethalin, trifluralin, amiprophos-methyl, butamiphos, dithiopyr, thiazopyr, propyzamide (pronamide), tebutam, propyzamide (pronamide), tebutam, chlorthal-dimethyl (DCPA), acetochlor, alachlor, butachlor, dimethachlor, dimethenamid, metazachlor, metolachlor, pethoxamid, pretilachlor, propachlor, propisochlor, thenylchlor, diphenamid, napropamide, naproanilide, flufenacet, mefenacet, fentrazamide, anilofos, cafenstrole, piperophos, dichlobenil, chlorthiamide, chlorpropham, propham, carbetamide, and combinations thereof.

In embodiment 62, the invention provides a process according to any one of embodiments 59 to 61, wherein the herbicide is selected from amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-sodium, foramsulfuron, halosulfuron-methyl, imazosulfuron, iodosulfuron, mesosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, penoxsulam, bispyribac-sodium, pyribenzoxim, pyriftalid, pyrithiobac-sodium, pyriminobac-methyl, flucarbazone-sodium, propoxycarbazone-sodium, glyphosate, sulfosate, benazolin-ethyl, glufosinate-ammonium, bialaphos (bilanaphos), mesotrione, sulcotrione, isoxachlortole, isoxaflutole, benzofenap, pyrazolynate, pyrazoxyfen, Benzobicyclon, bromobutide, (chloro)-flurenol, Cinmethylin, Cumyluron, Dazomet, dymron (daimuron), methyl-dymron (methyl-dimuron), etobenzanid, fosamine, indanofan, metam, oxaziclomefone, oleic acid, pelargonic acid, pyributicarb, benefin (benfluralin), butralin, dinitramine, ethalfluralin, oryzalin, pendimethalin, trifluralin, amiprophos-methyl, butamiphos, dithiopyr, thiazopyr, propyzamide (pronamide), tebutam, propyzamide (pronamide), tebutam, chlorthal-dimethyl (DCPA), chlorpropham, propham, carbetamide, and combinations thereof.

In embodiment 63, the invention provides a process according to any one of embodiments 59 to 62, wherein the herbicide is selected from chlorpropham, propham, carbetamide, and combinations thereof; most preferably chlorpropham.

In embodiment 64, the invention provides a process for preparing a Dunaliella alga, or extract thereof; or a powdered Dunaliella alga, or extract thereof; according to any one of embodiments 24 to 30, wherein the process is as defined in any one of embodiments 53 to 63.

In embodiment 65, the invention provides the use of a Dunaliella alga, or extract thereof; or a powdered Dunaliella alga, or extract thereof; as defined in any one of embodiments 1 to 30; as a food colourant or food ingredient; or as a health supplement.

In embodiment 66, the invention provides the use of a Dunaliella alga, or extract thereof; or a powdered Dunaliella alga, or extract thereof; as defined in any one of embodiments 1 to 30; in a cosmetic composition.

In embodiment 67, the invention provides a Dunaliella alga, or extract thereof; or a powdered Dunaliella alga, or extract thereof; as defined in any one of embodiments 1 to 30; or a composition as defined in embodiment 31, for use in therapy.

### Definitions

The term ‘Dunaliella alga’ as used herein refers to the multiple strains of Dunaliella that produce carotenoids. Nomenclature of these strains has not historically been consistent.

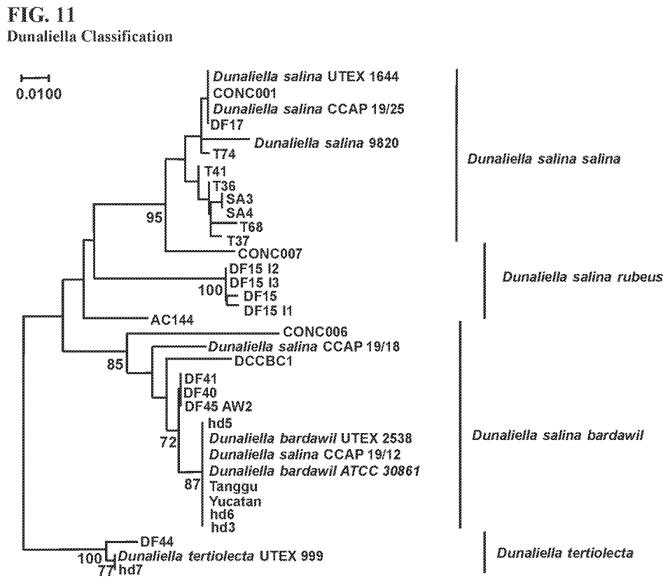

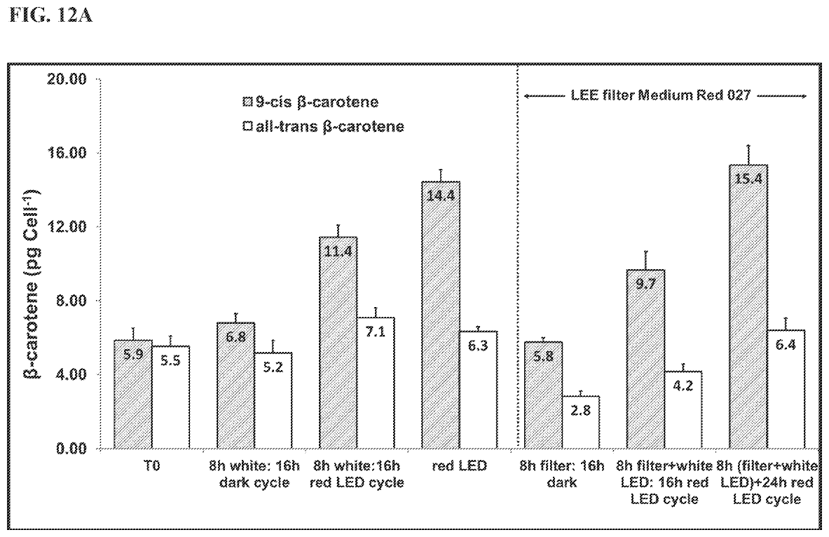

For example, Dunaliella bardawil is considered by some references to be a strain of Dunaliella salina, but is considered by others to be a different strain. FIG. 11 shows the grouping of Dunaliella strains by the Marine Biological Association (MBA), together with the nomenclature used herein. Preferably, the term ‘Dunaliella algae, as used herein refers to any strain of Dunaliella salina salina, Dunaliella salina rubeus, Dunaliella salina bardawil and Dunaliella tertiolecta, as classified in FIG. 11. Particularly preferred strains are PLY DF15 (CCAP 19/41), PLY DF17, PLY DF40 (CCAP 19/40), and UTEX2538.

The term ‘grown or cultivated under natural light or white light conditions’ as used herein, refers to Dunaliella algae growing, or specifically cultivated, in ponds, lakes, lagoons, raceways or closed vessels under natural light or under artificial white light.

The term ‘raceway’ as used herein, refers to a shallow pond that uses sunlight as the light source and paddlewheels to provide the flow to circulate algae, water and nutrients keeping the algae suspended in the water, and circulating them back to the surface on a regular frequency. The ponds are operated continuously with carbon dioxide or flue gas containing CO2 and nutrients are fed constantly or by batch to the ponds.

The term ‘cascade raceway’ as used herein, refers to a raceway which uses gravity instead of a paddlewheel to promote the mixing of the culture as it flows on the surface of inclined surfaces. After each cycle it is necessary to reposition the culture on the top part of the cascade through a pump or another device thus ensuring the flow cycle is closed.

The term ‘photobioreactor’ (PBR) as used herein, refers to a closed vessel or bioreactor, which incorporates some type of light source for photo- or mixo-trophic cultivation of algae. The light source is usually sunlight, but can also include artificial lighting. All essential nutrients must be introduced into the system to allow algae to grow and be cultivated. A photobioreactor can be operated in “batch mode” but it is also possible to introduce one or multiple continuous streams of process water containing nutrients, air and carbon dioxide. Temperature control (heating and cooling) are easily achievable. Many photobioreactor designs have been created and include vertical Green-wall flat panels (Green-walls, GW) comprising a thin layer of liquid (5-10 cm) contained in an aerated transparent plastic bag supported by a metal framework, and tubular photobioreactors, which consist of vertical or horizontally displayed transparent tubes, which can be stacked in groups to yield parallel fence-like vertical sets, and connected through piping accessories to a tank/degassing column, where most of the automation equipment is located, as well as the inlets and outlets for all the utilities. Culture mixing is ensured by pumping (in some cases also compressed air).

The term ‘increased content of’ as used herein, refers to an increase in the content of the carotenoid relative to the content found in Dunaliella algae which is grown or cultivated under natural conditions, i.e. under natural light or white light conditions and without herbicide treatment.

The term ‘early orange phase’ as used herein, refers to the growth phase that typifies the start of carotenogenesis, and is usually associated with the onset of stress related to deficiency in nitrate, sulfate, and phosphate in the culture media as well as high light intensity and high sodium chloride concentration. The carotenoid:chlorophyll ratio in cells is typically 3 or more.

The term ‘log growth phase’ as used herein refers to the period of algal growth characterized by cell doubling (also known as the logarithmic phase or exponential phase). The carotenoid:chlorophyll ratio in cells is typically around 1.

The term ‘powdered Dunaliella alga’ as used herein refers to a powdered product of Dunaliella alga which may be obtained by spray-drying or freeze-drying or any other method of dehydration.

The term ‘light of wavelength’ or ‘wavelength in the range of’ as used herein, refers to light having a wavelength of light emittance in the specified range by the source. For the avoidance of doubt the wavelength of light range includes either a single wavelength of light emittance within the specified range or any number of single wavelengths of light emittance within the specified range.

The term ‘herbicide’ as used herein refers to a composition which controls, suppresses or destroys plant growth. The herbicide may be defined by the mechanism of action, including phytoene desaturase inhibitors, phytochrome inhibitors, auxin-type (synthetic auxin) herbicides), cell division inhibitors, enolpyruvylshikimate 3-phosphate synthase enzyme (EPSPS) inhibitors, acetyl coenzyme A carboxylase (ACCase) inhibitors, acetolactate synthase (ALS) inhibitors, photostem II inhibitors, photostem I inhibitors, and 4-hydroxyphenylpyruvate dioxygenase (HPPD) inhibitors.

The invention is illustrated by the following examples.

### Example 1

Dunaliella algae were cultured in the laboratory in an ALGEM Environmental Modeling Labscale Photobioreactor (Algenuity, UK), at 25° C. Approximately 5×10⁷ cells were inoculated in 500 ml Modified Johnsons Medium (Borowitzka, Algal growth media and sources of cultures, in Microalgal Biotecnology. Borowitzka, L. J. (Eds.), 1988, pp. 456-465) containing 1.5 M NaCl and placed under a cycle of 12 h/12 h Light/Dark conditions. Cells were grown under 200 μmol photons·m⁻²·s⁻¹ of white LED light. In one set of experiments cells were cultivated to a cell density of ˜0.5×10⁶ cells mL⁻¹ and then the cultures were diluted with fresh medium to a cell density of ˜0.2×10⁶ cells mL⁻¹. Under these conditions cells were in the early orange phase of growth but not placed under nutrient stress. The cultures were then exposed to either white LED light, red LED light, or blue LED light at the same light intensity of 1000 μmol m⁻² s⁻¹, or white LED light of 1000 μmol m⁻² s⁻¹ covered with one of three different red filters (filter 26 Bright red, 27 Medium Red and 787 Marius Red supplied by LEE Filters) for 48 hours. Each light condition was set up in at least triplicate. Dunaliella algae used in these experiments were the following strains:

PLY DF15, classified as D salina rubeus (and held by the Marine Biological Association Culture Collection, origin Israel) and also classified as CCAP 19/41 and held by the Culture Collection of Algae and Protozoa (CCAP).

PLY DF17, classified as D. salina salina (held by the Marine Biological Association Culture Collection, origin Israel)

PLY DF40, classified as D. salina bardawil (held by the Marine Biological Association Culture Collection, origin Spain) and also classified as D. salina CCAP 19/40 and held by the Culture Collection of Algae and Protozoa (CCAP).

UTEX 2538, classified as D. salina bardawil (Culture Collection of Algae and Protozoa (CCAP))

### Example 2

In a second set of experiments cells were cultivated to the log phase of growth and then kept in the dark for 36 hours for dark adaption. After dark adaption, the cultures were exposed to continuous blue or red LED light at different light intensities of 200, 500, and 1000 μmol photons·m⁻²·s⁻¹ for 48 hours. Each growth condition was set up in at least triplicate.

### Example 3

In a third set of experiments, Dunaliella algae were cultivated at 25° C. under 200 μmol photons·m⁻²·s⁻¹ of white LED light to log phase and then kept in the dark for 36 hours for dark adaption. Then the cultures were exposed to continuous blue or red LED light at the light intensity of 1000 μmol photons·m⁻²·s⁻¹ at 15° C. compared to 25° C. for 48 hours.

Cell concentration: Cell concentration was determined by counting the number of cells in culture broth using a haemocytometer, after fixing with 2% formalin. Samples were taken at 0, 24 and 48 hours to determine the cellular contents of carotenoids and chlorophyll and the composition of the carotenoids.

Pigment analysis: 1 ml culture broth was centrifuged at 3,000 g in a bench-top centrifuge for 5 min. to harvest the algal biomass and pigments were extracted from the biomass using 1 ml of 80% (v/v) acetone. After clarification at the centrifuge, the absorbance of the acetone extract was measured at 480 nm in a spectrophotometer. The content of total carotenoids was calculated according to Strickland & Parsons (Strickland, J. & Parsons, T. R., 1972. A practical handbook of seawater analysis 2nd ed., Fish Res Board Can Bull.): Total Carotenoids (μg·ml⁻¹)=4.0*Abs₄₈₀ₙₘ, where Abs₄₈₀ₙₘ is the absorbance of 80% acetone extract measured at 480 nm.

Chlorophyll a, b and total Chlorophyll were evaluated by measuring the absorbance of the acetone extract at 664 nm and 647 nm and calculated according to Porra, R. J., Thompson, W. A. & Kriedemann, P. E., 1989. Determination of accurate extinction coefficients and simultaneous equations for assaying chlorophylls a and b extracted with four different solvents: verification of the concentration of chlorophyll standards by atomic absorption spectroscopy. Biochimica et Biophysica Acta (BBA)-Bioenergetics, 975(3), pp. 384-394:

Chl a (μg·ml⁻¹)=(12.25*Abs₆₆₄ₙₘ)−(2.55*Abs₆₄₇ₙₘ);

Chl b (μg·ml⁻¹)=(20.31*Abs₆₄₇ₙₘ)−(4.91*Abs₆₆₄ₙₘ),

Total Chl (μg·ml⁻¹)=Chl a (μg·ml⁻¹)+Chl b (μg·ml⁻¹),

where Abs₆₄₇ₙₘ and Abs₆₆₄ₙₘ refer to the absorbance of the 80% acetone extract measured at 664 nm and 647 nm respectively.

The compositions of pigments were analysed using an HPLC with fitted with diode-array detection (DAD). 15 ml culture broth was centrifuged at 3,000 g in a bench-top centrifuge for 5 min. to harvest the algal biomass as before. Algal biomass was extracted with 10 ml MTBE-MeOH (20:80), after sonication for 20 s. Each sample was clarified by centrifugation at 3,000 g for 10 min then filtered through a 0.45 μm filter into amber HPLC vials. The samples were analysed using a YMC30 250×4.9 mm I.D S-5μ HPLC column with DAD at 25° C., and isocratic elution with 80% methanol: 20% MTBE, flow rate of 1 mL min⁻¹, pressure of 90 bar. The quantities of β-carotene in the biomass were estimated using a β-carotene standard curve prepared with synthetic all-trans β-carotene from Sigma, and the quantities of phytoene and α-carotene, with reference to standards of each from Sigma. Each experiment was carried out in at least triplicate.

### Example 4

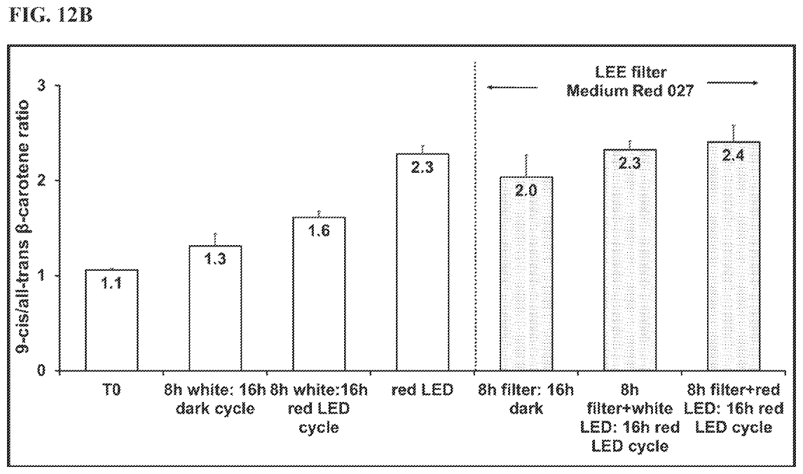

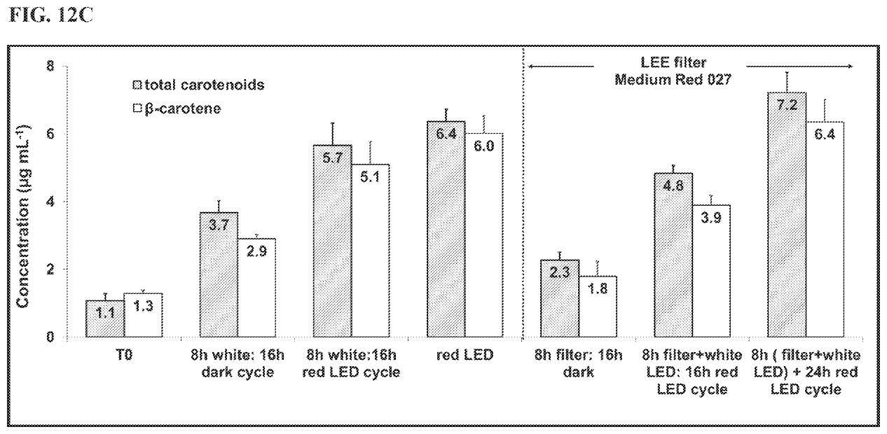

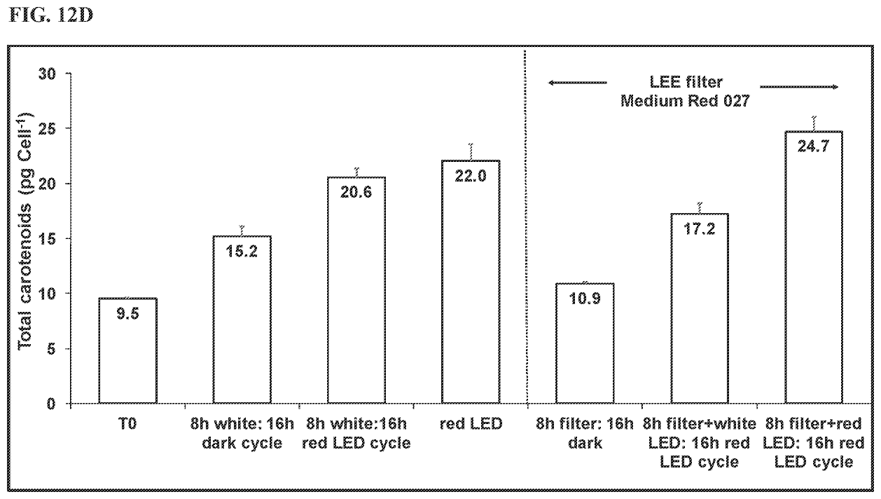

Treatment of D. salina cultures with red light included in the cultivation cycle was observed to increase both the ratio of 9-cis to all-trans β-carotene and the amount of carotenoid compared to cultivation under a white:dark light cycle, with the greatest increases occurring with continuous red light, whether applied with red LED or with red filters. Compensation for the intensity of light emitted by LED lights may be required when red filters are applied as covers to LED lights. The results are presented in FIGS. 12A-12D. All treatments with red light included in the cycle increased both the ratio of 9-cis to all-trans β-carotene compared to the natural condition and the amount of carotenoid, with the greatest increases occurring with continuous red light, whether applied with red LED or with red filters, but red filters applied to LED lights reduces the light intensity emitted and consequently the cellular productivity.

### Example 5

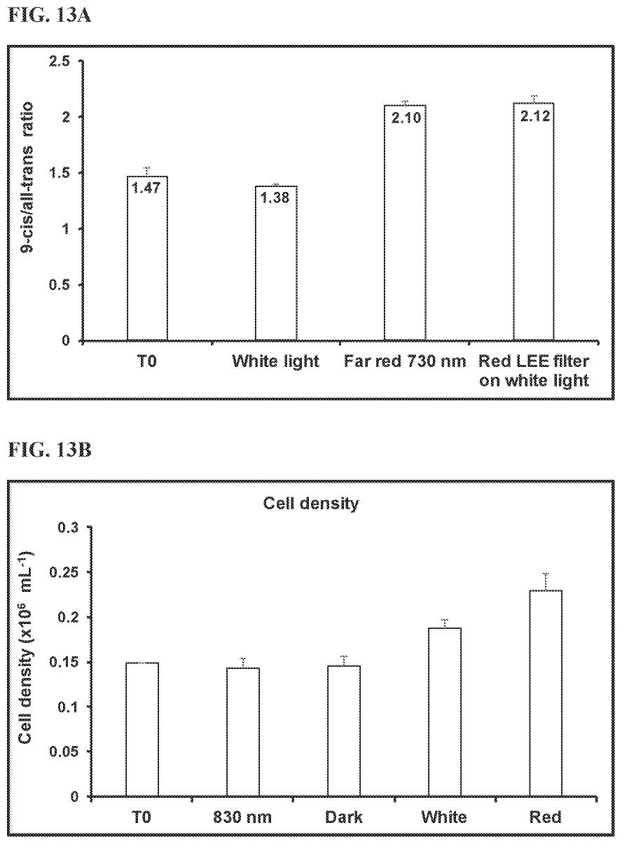

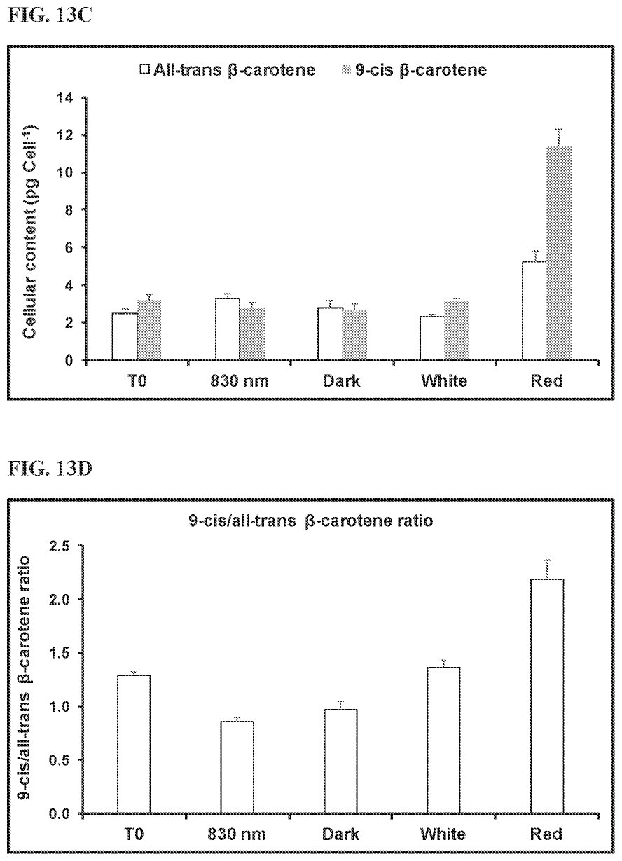

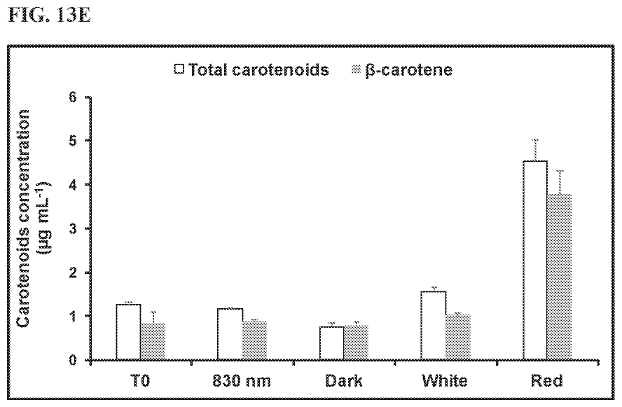

Treatment of D. salina cultures with far-red light of 730 nm was found to be as effective in increasing β-carotene production and the 9-cis/all-trans ratio as red light transmitted by Lee Filter 027 (600-700 nm). The carotenoids, 9-cis/all-trans ratio and chlorophyll content of cultures under far red and red light were identical. Both far red light and red light increased the 9-cis/all-trans ratio from ˜1.5 to ˜2.0 compared to white light alone. By contrast with LED light of wavelength 830 nm applied for 3 days, the cells did not divide, as was also found for cells placed in the dark for 3 days. The 9-cis/all-trans β-carotene ratio decreased for cells placed in the dark or treated with 830 nm light compared to untreated cells and the yield of carotenoids and β-carotene also slightly decreased. The results are presented in FIGS. 13A-13E.

### Example 6

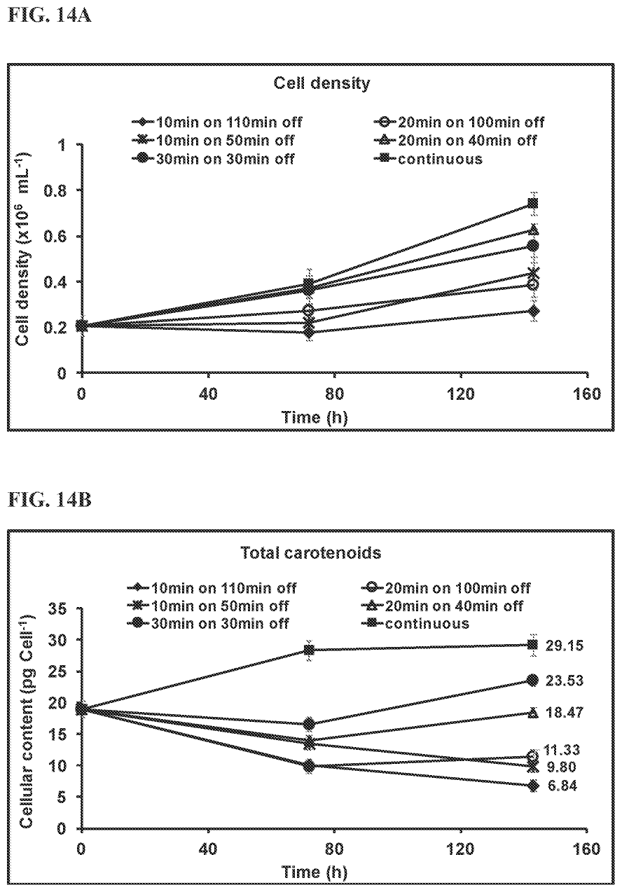

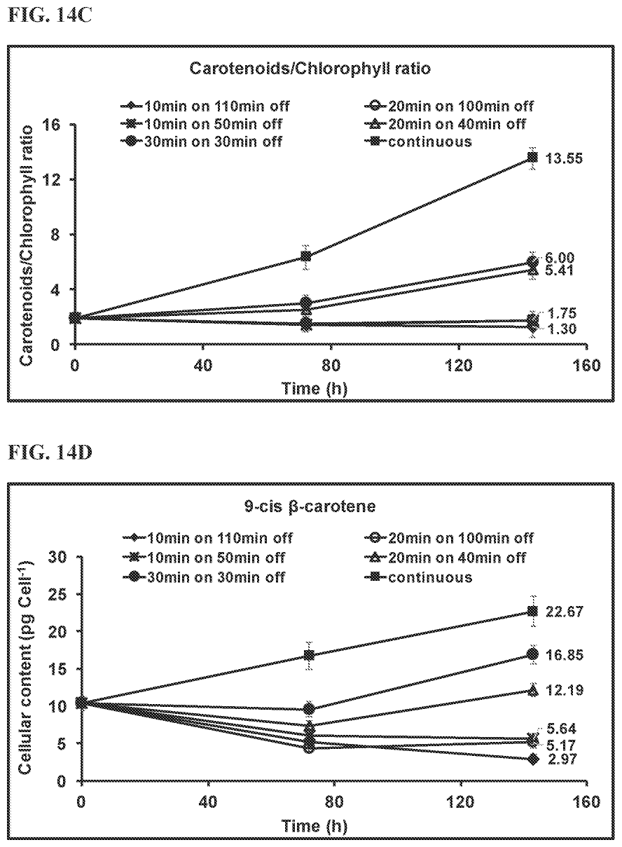

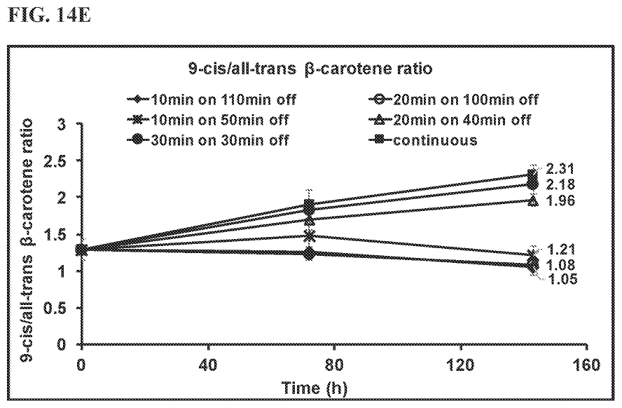

Treatment of D. salina cultures with red light-dark cycles of increasing red light cycle duration was found to increase cell density, total carotenoids and 9-cis: all-trans ratio, with the greatest effect being meted with continuous red light. 9-cis-β-Carotene content was found to continuously increase with continuous red light for 140 h, whereas total carotenoid content showed no further increase after 72 h, which may reflect decreasing cellular synthetic capacity, since total chlorophyll content declines continuously in continuous red light for 140 h. Results are presented in FIGS. 14A-14E.

### Example 7

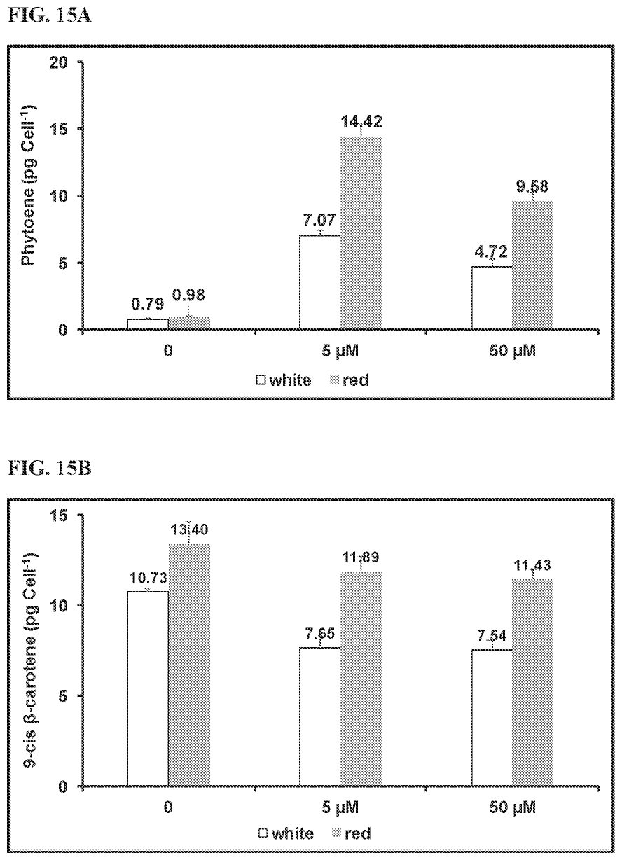

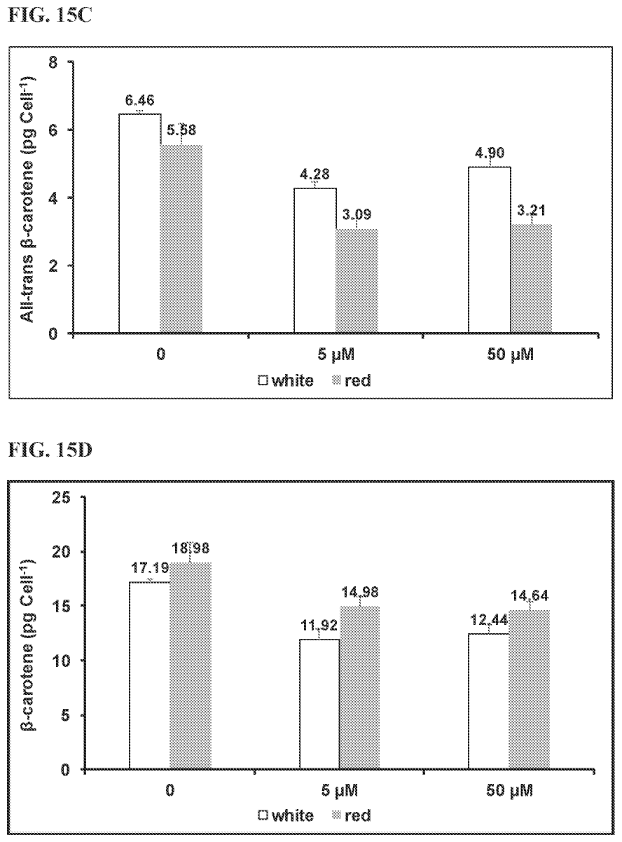

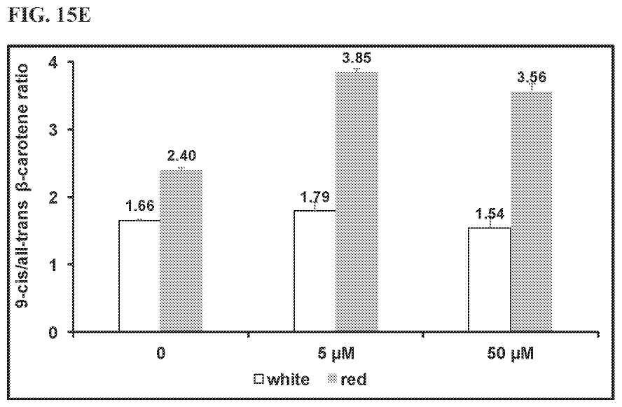

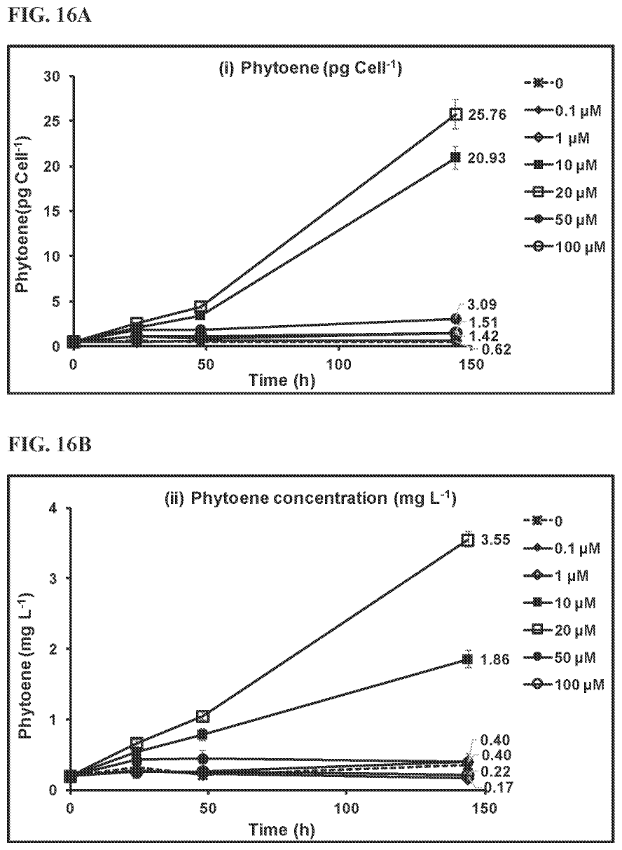

Treatment of D. salina cultures cultivated under red light with phytoene desaturase inhibitor herbicides was found to result in a significantly higher amount of phytoene when compared to cultivation under white light. Results are presented in FIGS. 15A-15E.

### Example 8

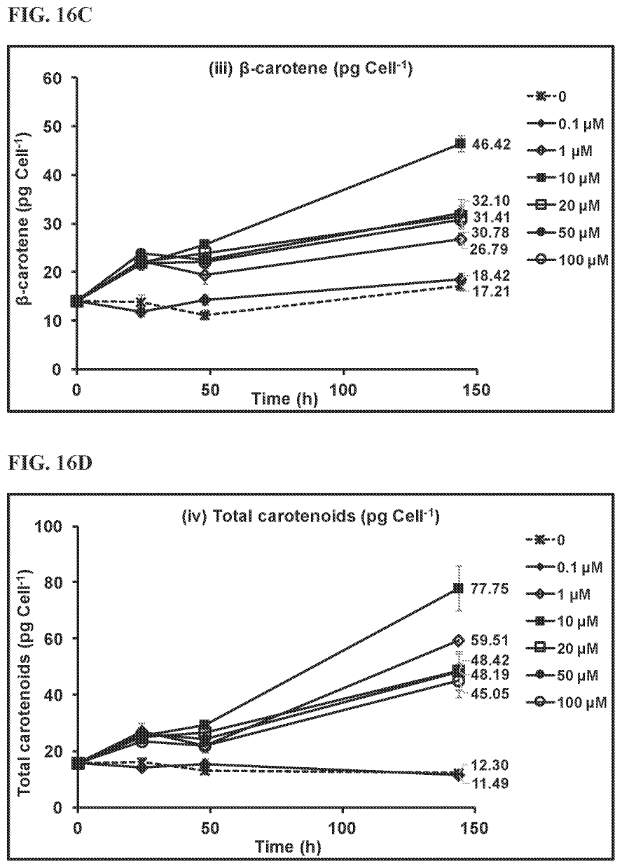

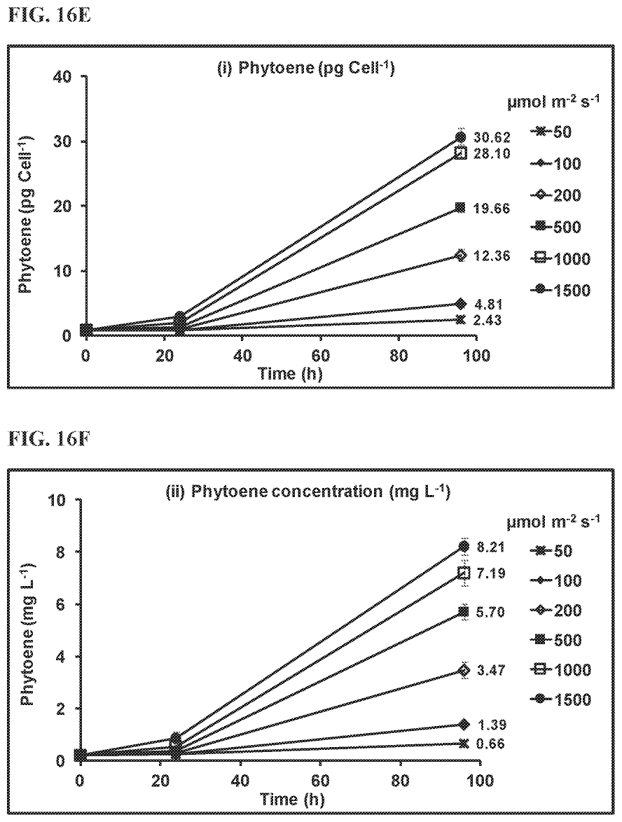

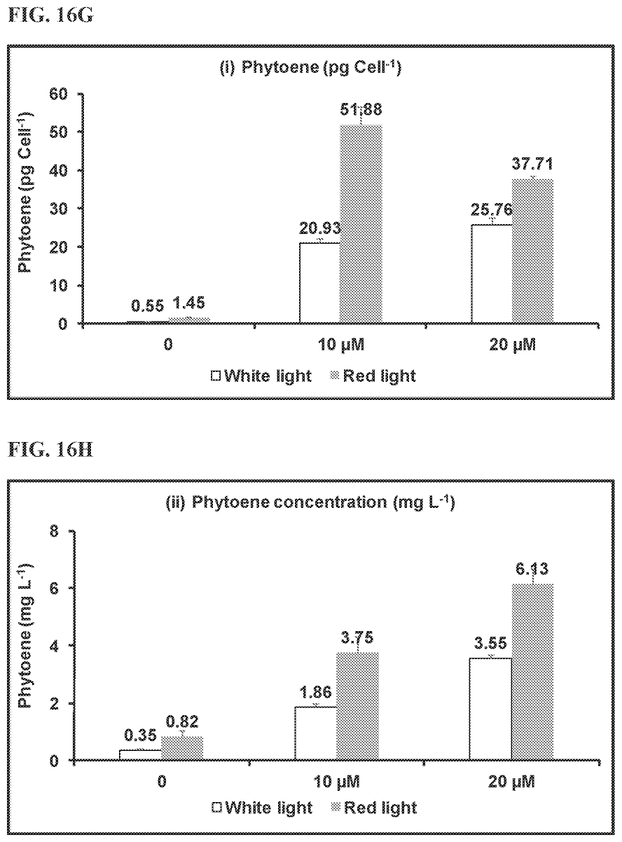

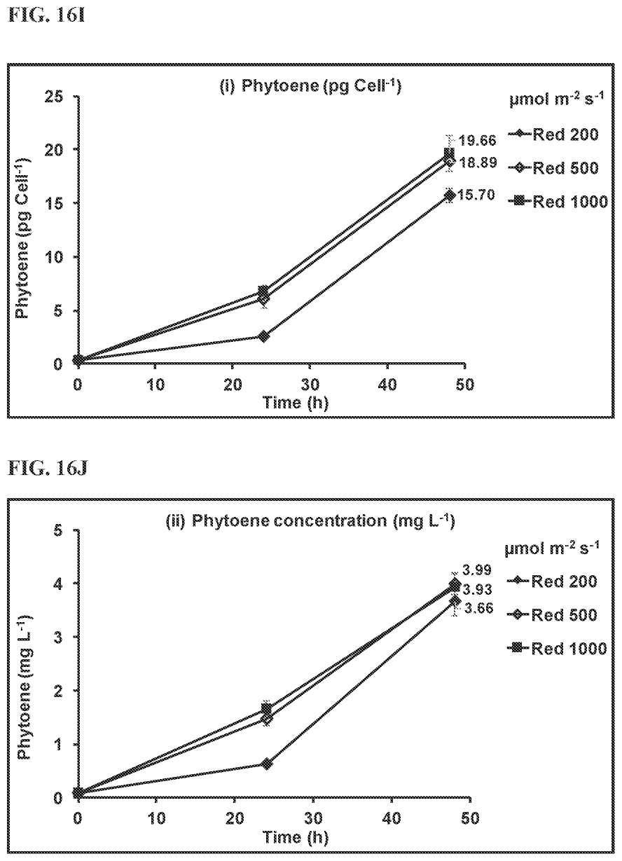

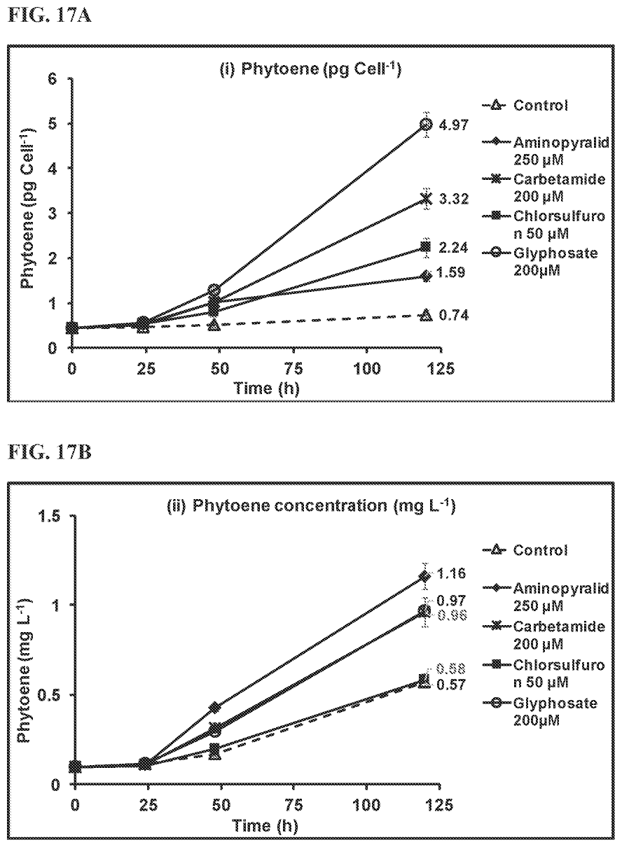

D. salina cultures were treated with herbicides which inhibit cell division, such as chlorpropham (CIPC), aminopyralid, carbetamide and chlorsulfuron, or with phytochrome inhibitors such as glyphosate. The content of both phytoene and phytofluene as well as the content of coloured carotenoids were found to have increased when D. salina is cultured in the presence of the herbicides, and cultivation under red light was found to magnify the effects. Results are presented in Table 4 and FIGS. 16A-16J.

### Example 9

D. salina cultures were treated with chlorpropham. The cellular content of colorless carotenoids was found to increase by more than 30-fold and the yield of the colorless carotenoids was found to increase more than 10-fold compared to untreated cultures. The optimal concentration range of chlorpropham added to the cultures was determined to be 10-50 μM. Cell density stopped increasing once chlorpropham was added, and carotenoids in particular phytoene and phytofluene started to accumulate. Chlorpropham is preferably added to the cultures when a high cell density is achieved.

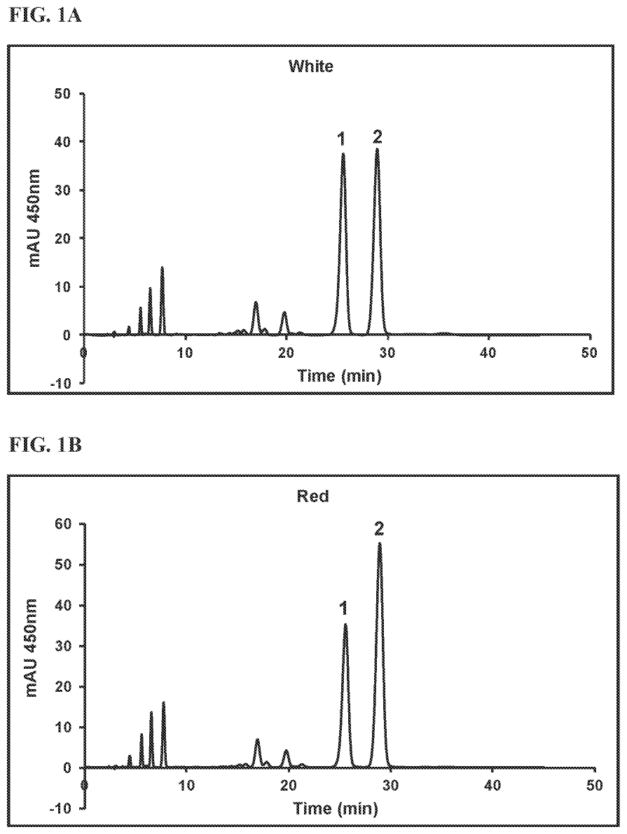

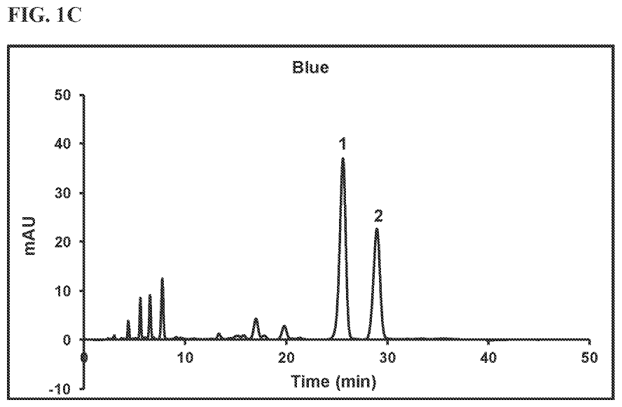

FIGS. 1A-1C. HPLC profiles of carotenoid extracts from Dunaliella salina exposed to continuous white light (FIG. 1A), red light (FIG. 1B) and blue light (FIG. 1C) at 1000 μmol m⁻² s⁻¹ for 48 hours after initial growth to early orange phase of the growth cycle in white light. Peak 1: all-trans β-carotene; peak 2: 9-cis-β-carotene. The Figure shows absorbance profile at 450 nm.

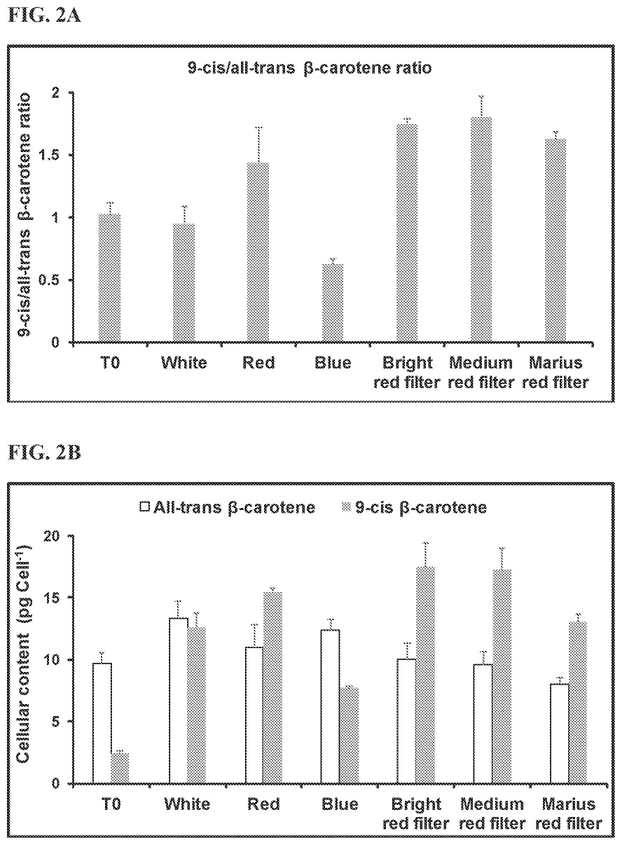

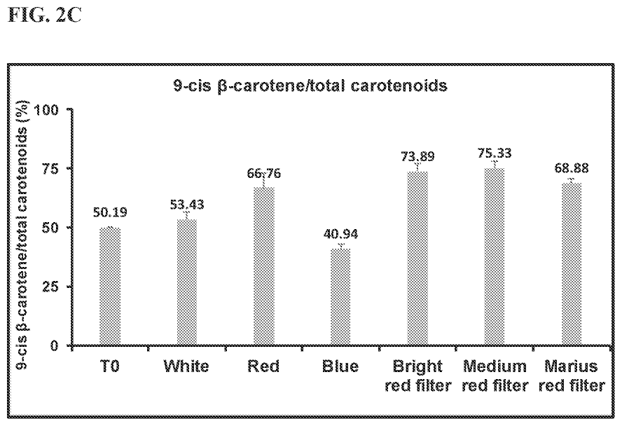

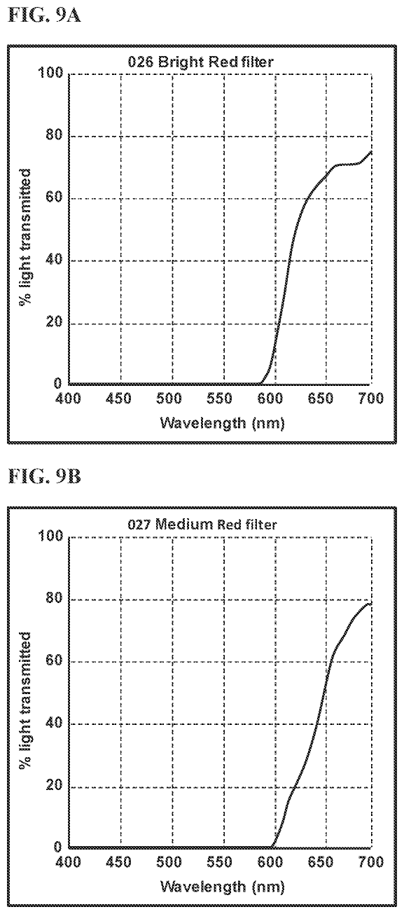

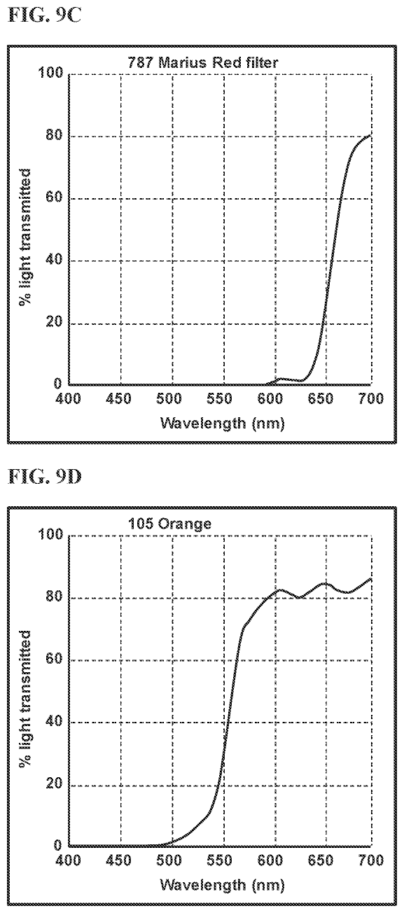

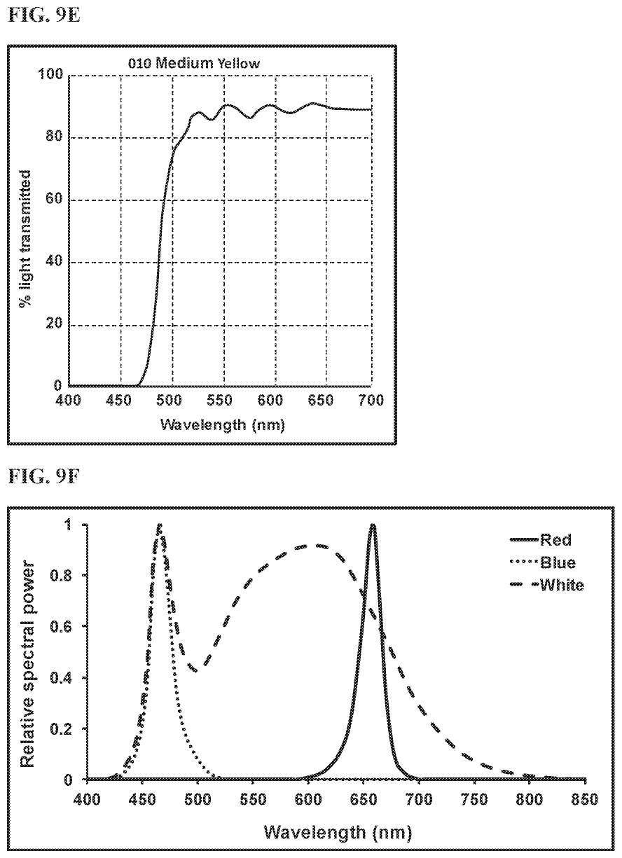

FIGS. 2A-2C. Effect of different light treatments on the ratio of 9-cis and all-trans β-carotene (FIG. 2A); the cellular content of all-trans β-carotene and of 9-cis β-carotene (FIG. 2B); and the amount of 9-cis β-carotene as a % of the total amount of carotenoids (FIG. 2C) in Dunaliella salina when cultivated to early orange phase until light treatment (T0) and then subjected to different light treatments for 48 hours. Cells were cultured in light:dark 12 h:12 h in incubators with white light to early orange phase (cell density of ˜0.5×10⁶ cells mL⁻¹; carotenoid:chlorophyll ratio ˜3) and then cultures were diluted with fresh medium to a cell density of ˜0.2×10⁶ cells mL⁻¹ (No nutrient stress). The cultures were then exposed to either white LED light, red LED light, or blue LED light at the same light intensity of 1000 μmol m⁻² s⁻¹, or white LED light of 1000 μmol m⁻² s⁻¹ covered with one of three different red filters (Lee filter 26 Bright red, 27 Medium Red and 787 Marius Red, see FIGS. 9A-9F) for 48 hours. Each light condition was set up in at least triplicate. The data show clearly the increase in 9-cis: all-trans β-carotene ratio and increase in 9-cis β-carotene as a % of total carotenoids after exposure to red light. Red light applied using filters may vary in total light intensity delivered to cells (see FIGS. 9A-9F for examples of transmission % using the filters illustrated). This effect is most notable with use of the 787 Marius Red filter, which cut out approximately 98% of the light intensity applied such that cells received only approximately 10-17 μmol m⁻² s⁻¹ light intensity of the red light. The effect of red light delivered with the 787 Marius Red filter still prevailed to increase the ratio of 9-cis-: all-trans β-carotene and the amount of 9-cis β-carotene as a % of the total amount of carotenoids.

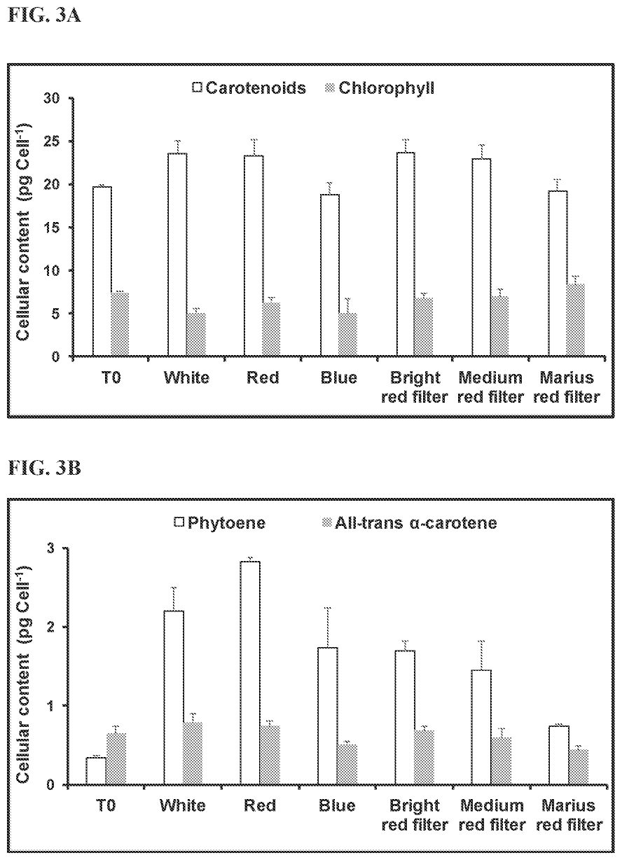

FIGS. 3A-3B. Effect of different light treatments on the cellular content of total carotenoids and chlorophyll (FIG. 3A), and of phytoene and of α-carotene (FIG. 3B) in Dunaliella salina when cultivated to early orange phase until light treatment (T0) and then subjected to different light treatments for 48 hours.

Cells were cultured in light:dark 12 h:12 h in incubators with white light to early orange phase (cell density of ˜0.5×10⁶ cells mL⁻¹; carotenoid:chlorophyll ratio ˜3) and then cultures were diluted with fresh medium to a cell density of ˜0.2×10⁶ cells mL⁻¹ (no nutrient stress). The cultures were then exposed to either white LED light, red LED light, or blue LED light at the same light intensity of 1000 μmol m⁻² s⁻¹, or white LED light of 1000 μmol m⁻² s⁻¹ covered with one of three different red filters (Lee filter 26 Bright red, 27 Medium Red and 787 Marius Red, see FIGS. 9A-9F) for 48 hours. Each light condition was set up in at least triplicate.

These data show that the cellular content of chlorophyll and in turn phytoene and α-carotene may vary to compensate for reduced light availability using filters (see FIGS. 9A-9F for examples of transmission % using the filters illustrated). This effect is most notable with use of the 787 Marius Red filter, which cut out approximately 98% of the light intensity applied such that cells received only approximately 10-17 μmol m⁻² s⁻¹ light intensity of the red light.

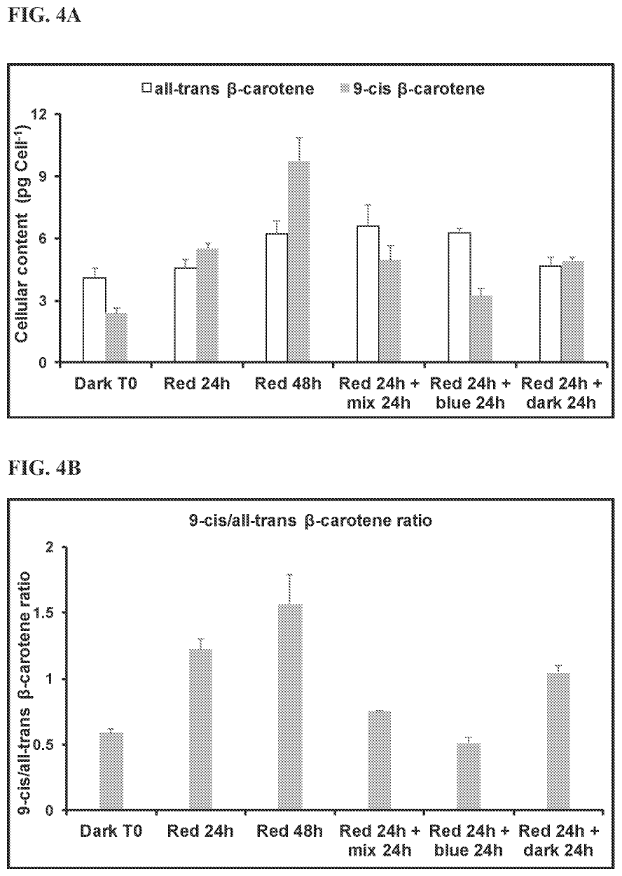

FIGS. 4A-4B. Effect of different light treatments on the cellular content of 9-cis β-carotene and all-trans β-carotene (FIG. 4A) and the ratio of 9-cis and all-wans β-carotene (FIG. 4B) in Dunaliella salina when cultivated to mid-log phase of growth until light treatment (T0) and then subjected to different light treatments. Cells were cultured in light:dark 12 h:12 h white light growth regime to mid-log phase of the growth cycle (0.1-0.2×10⁶ cells mL⁻¹; carotenoid:chlorophyll ratio ˜1) then transferred to a further 24 h dark (Dark TO) before being exposed to continuous red LED light at 1000 μmol m⁻² s⁻¹ for 24 hours (Red 24 h). Cells were then treated for 24 hours under either red light (Red 48), a mix of 1:1 red and blue light (Red 24 h+mix 24 h), blue light (Red 24 h+blue 24 h) at the same light intensity of 1000 μmol m⁻² s⁻¹ or dark (Red 24 h+dark 24 h). Each light condition was set up at least in triplicate. These data show clearly a 4-fold increase in 9-cis-β-carotene content after exposure to 48 h red light (9.75±1.09 pg cell⁻¹) compared to dark-adapted cells (2.39±0.22 pg cell⁻¹). The ratio of 9-cis-β-carotene: all-trans β-carotene after 48 h red LED light was 1.58 whereas that for dark-adapted cells was 0.59 (see Table 2). In the cycle of red light 24 h followed by dark 24 h, the amount of 9-cis-β-carotene was maintained constant, but in the cycle of red light 24 h followed by blue light 24 h, approximately 35% of β-cis-β-carotene was lost. This effect was negated by using the 1:1 red/blue light mix instead of blue light alone.

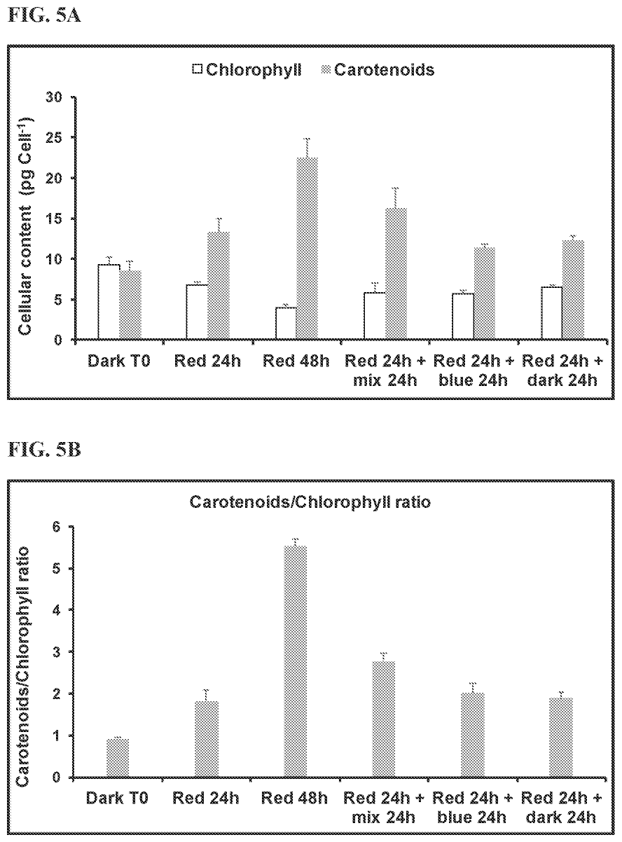

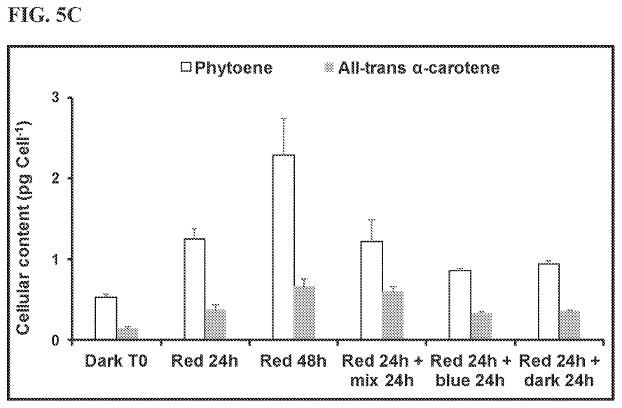

The total carotenoid content increased from 8.58±1.09 pg cell⁻¹ (dark-adapted cells) to 22.47±2.34 pg cell⁻¹ after treatment with red light for 48 h (2.6-fold increase) (see FIGS. 5A-5C).

FIGS. 5A-5B. Effect of different light treatments on the cellular content of chlorophyll and total carotenoids (FIG. 5A) and the ratio of total carotenoids to total chlorophyll (FIG. 5B) and on the cellular content of phytoene and all-trans-α-carotene in Dunaliella salina. Cells were cultured in light:dark 12 h:12 h white light growth regime to mid-log phase of the growth cycle (0.1-0.2×10⁶ cells mL⁻¹; carotenoid:chlorophyll ratio ˜1) then transferred to a further 24 h dark (Dark T0) before being exposed to continuous red LED light at 1000 μmol m⁻² s⁻¹ for 24 hours (Red 24 h). Cells were then treated for 24 hours under either red light (Red 48), a mix of 1:1 red and blue light (Red 24 h+mix 24 h), blue light (Red 24 h+blue 24 h) at the same light intensity of 1000 μmol m⁻² s⁻¹ or dark (Red 24 h+dark 24 h). Each light condition was set up at least in triplicate.

The total carotenoid content increased from 8.58±1.09 pg cell⁻¹ (dark-adapted cells) to 22.47±2.34 pg cell⁻¹ after treatment with red light for 48 h (2.6-fold increase). The chlorophyll content decreased under these conditions such that the ratio of carotenoids:chlorophyll increased from 2 in white light on exposure to red light for 24 h, to 5.5.

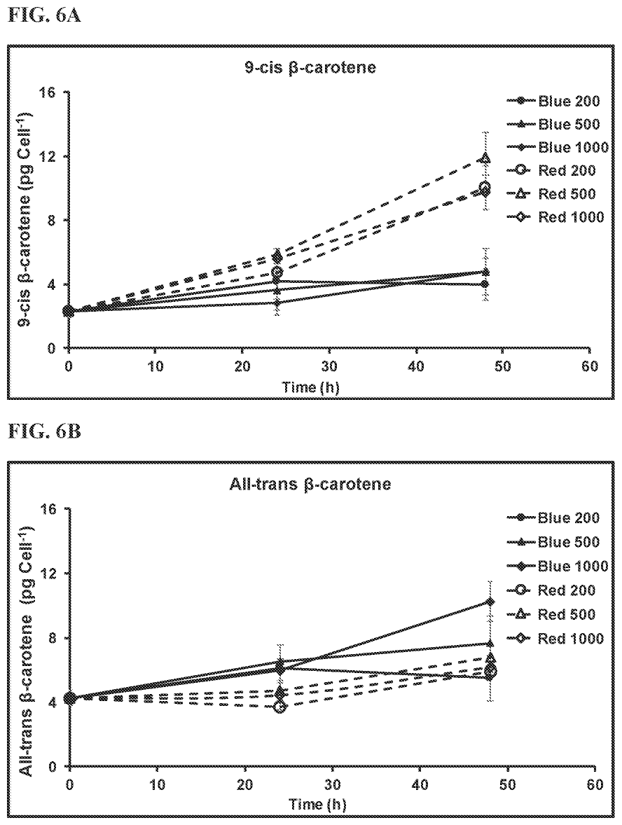

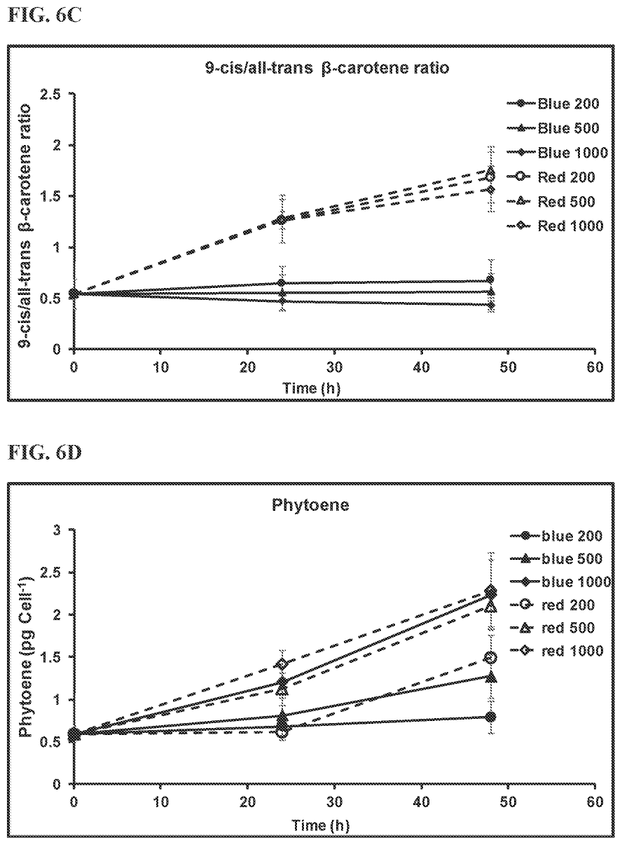

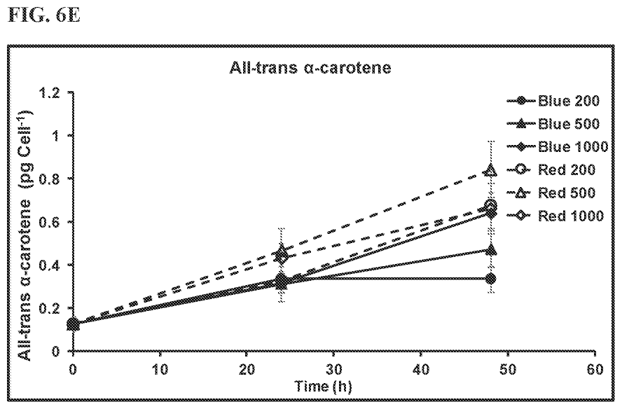

FIGS. 6A-6D. Cellular content of 9-cis β-carotene (FIG. 6A), all-trans β-carotene (FIG. 6B), the ratio of 9-cis and all-trans β-carotene (FIG. 6C), the content of phytoene (FIG. 6D) and the content of all-trans-α-carotene in Dunaliella salina cells treated with either continuous blue or red LED light at three different light intensities of 200, 500 and 1000 μmol m⁻² s⁻¹ for 48 hours. Cells were cultured in light:dark 12 h:12 h white light growth regime to mid-log phase of the growth cycle (0.1-0.2×10⁶ cells mL⁻¹; carotenoid:chlorophyll ratio ˜1) then transferred to a further 24 h dark (Dark T0) before exposure. Each light condition was set up at least in triplicate. (See also Table 5).

These data show that red LED light specifically enhances production of 9-cis-β-carotene relative to all-trans-β-carotene. Furthermore, the effect on 9-cis-β-carotene and on all-trans-β-carotene is independent of light intensity.

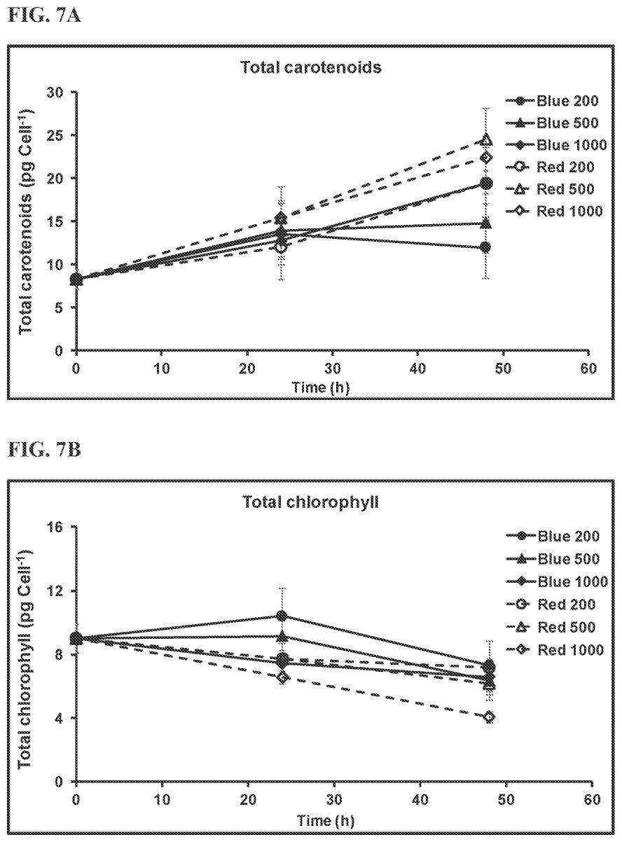

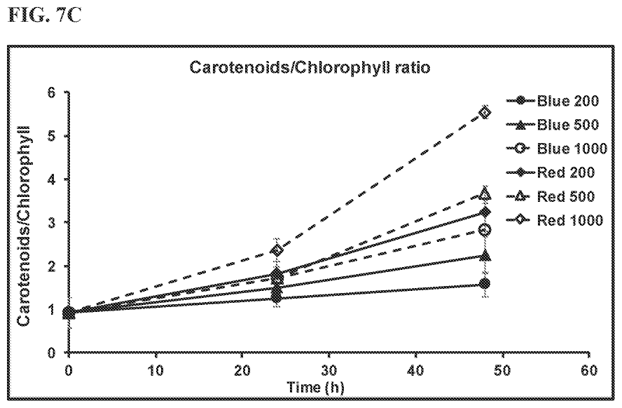

FIGS. 7A-7C. Cellular content of total carotenoids (FIG. 7A), total chlorophyll (FIG. 7B) and the ratio of total carotenoids to total chlorophyll (FIG. 7C) in Dunaliella salina cells treated with either continuous blue or red LED light at three different light intensities of 200, 500 and 1000 μmol m⁻² s⁻¹ for 48 hours. Cells were cultured in light:dark 12 h:12 h white light growth regime to mid-log phase of the growth cycle (0.1-0.2×10⁶ cells mL⁻¹; carotenoid:chlorophyll ratio ˜1) then transferred to a further 24 h dark (Dark T0) before exposure. Each light condition was set up at least in triplicate. These data show that red LED light specifically enhances production of total carotenoids.

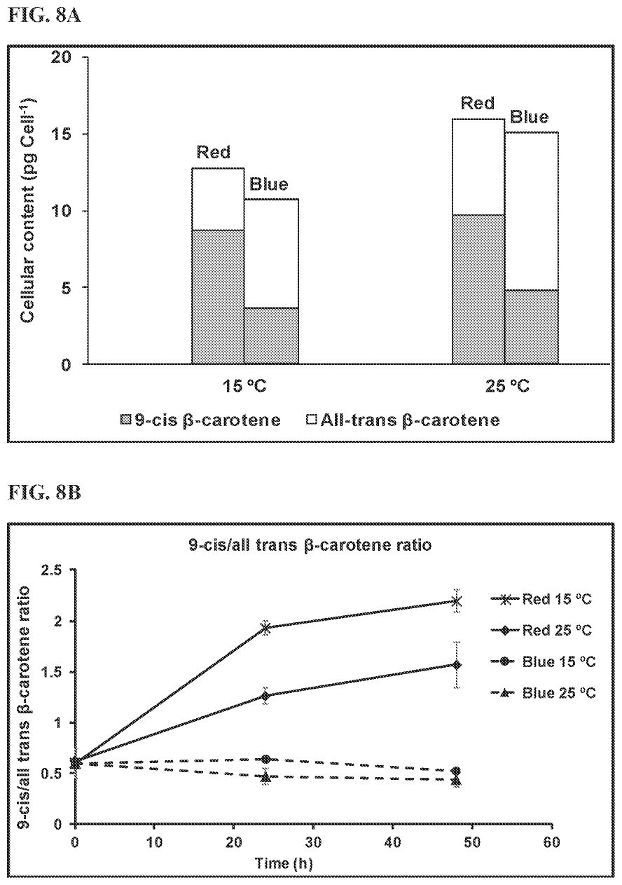

FIGS. 8A-8B. Effect of temperature on cellular content of 9-cis β-carotene and all-trans β-carotene (FIG. 8A) and the ratios of 9-cis and all-trans β-carotene (FIG. 8B) in Dunaliella salina cells exposed to red or blue LED light. Cells were cultured in light:dark 12 h:12 h white light growth regime to mid-log phase of the growth cycle (0.1-0.2×10⁶ cells mL⁻¹) then transferred to a further 24 h dark (Dark T0) before exposure to either continuous blue or red LED light at 1000 μmol m⁻² s⁻¹ for 48 hours at 15° C. or 25° C. for 48 hours. Each light condition was set up at least in triplicate. Reduction by 10° C. reduced the cellular content of all-trans-β-carotene but the cellular content of 9-cis-β-carotene was maintained and consequently the ratio of 9-cis: all-trans β-carotene increased to 2.2. (Compare FIGS. 4A-4B).

FIGS. 9A-9E: The light transmission (Y %) for each wavelength (nm) of typical filters that may be used to transmit red light, such as: (from Lee Filters) 26 Bright red (Transmission 8.6%), 27 Medium Red (Transmission 3.6%), 787 Marius Red (Transmission 1.0%). Filters that eliminate blue light will also be effective, such as: (from Lee Filters), 105 Orange (Transmission 41.3%), and 010 Medium Yellow (Transmission 86.5%). FIG. 9F shows the typical relative spectral power distribution of white, blue and red LED lights.

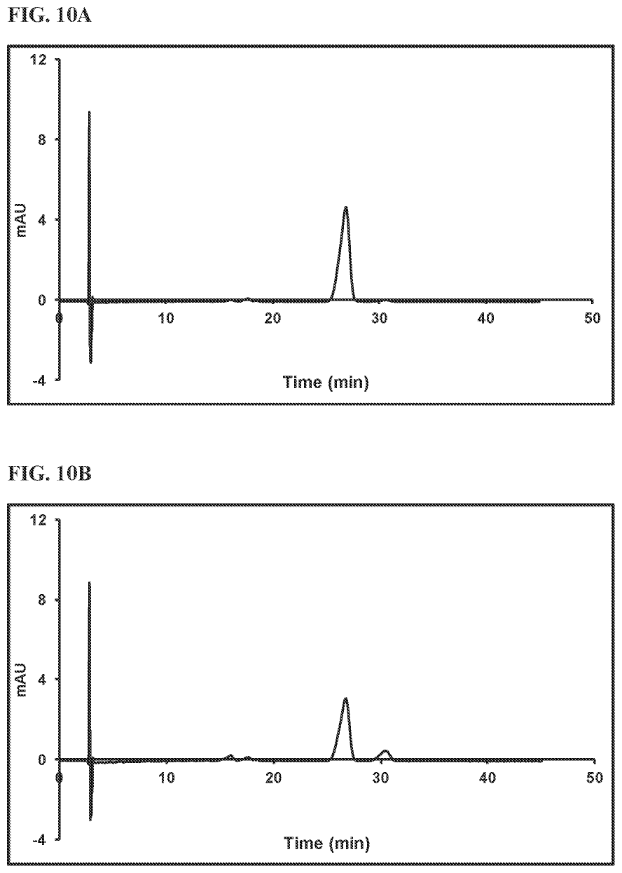

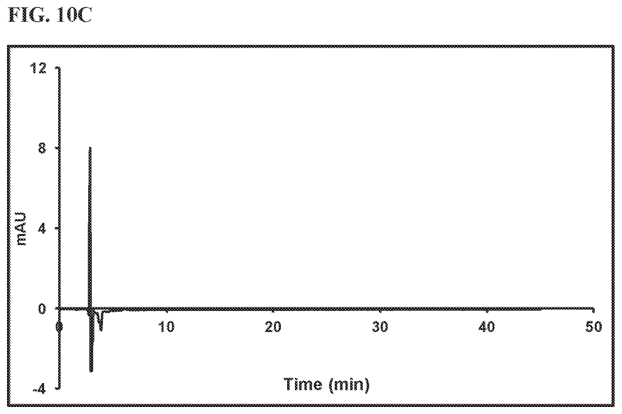

FIGS. 10A-10C: Effect of red and blue LED light on all-trans β-carotene. (FIG. 10A) Red light under nitrogen; (FIG. 10B) Red light in air; (FIG. 10C) Blue light in air. All-trans-β-carotene (Sigma) was dissolved in chloroform to a final concentration of 2.4 μM and vials were thoroughly flushed with either nitrogen or air, sealed and incubated for 24 h at 25° C. under LED lights. (FIG. 10A) red, nitrogen; (FIG. 10B), red, air; (FIG. 10C) blue, nitrogen or air. The same results as (FIG. 10A) were obtained for dark under nitrogen or air. In (FIG. 10B) 40% destruction of all-trans β-carotene was recorded in red light under air, whereas in (FIG. 10C) in blue light, all-trans β-carotene was fully destroyed within the same time frame. In (FIG. 10A) (red light under nitrogen) no reaction of β-carotene was detected. These data show that blue light is more damaging to all-trans β-carotene than is red light.

FIG. 11: Classification of Dunaliella strains (unpublished) as provided by the Director of The Marine Biological Association Culture Collection, Citadel Hill Plymouth PL 1 2PB.

FIGS. 12A-12D: Cellular content of (FIG. 12A) 9-cis β-carotene and all-trans β-carotene, (FIG. 12B) 9-cis/all-trans ratio (FIG. 12C) yield of carotenoids (μg ml⁻¹) and (FIG. 12D) cellular content of total carotenoids of D. salina cultures grown under different light cycles. T0, amounts at time 0.

Cultures of D. salina were grown to mid-log phase and then exposed to different 24 h cycles of light treatment applied for 3 days. Biomass was harvested at mid-day on the 3^{rd} day for analysis by HPLC. The cycles were as follows:
(1) 8 h white: 16 h dark cycle, 8 h white light (500 μmol m⁻² s⁻¹) followed by 16 h dark to simulate a day-night cycle, for 72 h.
(2) 8 h white: 16 h red LED cycle, 8 h white light (500 μmol m⁻² s⁻¹) followed by 16 h red LED light (500 μmol m⁻² s⁻¹), for 72 h.
(3) Continuous red LED, red LED light (500 μmol m⁻² s⁻¹), for 72 h.
(4) 8 h LEE filter:16 h dark, LEE filter medium red 027 covered over white light (500 μmol m⁻² s⁻¹) for 8 h, followed by 16 h dark, for 72 h.
(5) 8 h LEE filter+white LED: 16 h red LED cycle: LEE filter medium red 027 covered over white LED light (500 μmol m⁻² s⁻¹) for 8 h, and red LED light (500 μmol m⁻² s⁻¹) for 16 h, for 72 h.
(6) 8 h LEE filter+white LED+24 h red LED cycle: LEE filter medium red 027 covered over white light (500 μmol m⁻² s⁻¹) for 8 h, together with red LED light (500 μmol m⁻² s⁻¹) for 24 h, for 72 h.

FIGS. 13A-13E: The effect of red light and far-red light of 730 nm applied to D. salina cultures for 48 h on the ratio of 9-cis: all-trans β-carotene (FIG. 13A), and of light of 830 nm on cell density (FIG. 13B), all-trans- and 9-cis β-carotene (FIG. 13C), on the ratio of 9-cis: all-trans β-carotene (FIG. 13D) and total carotenoids and β-carotene (FIG. 13E).

Cultures of D. salina were grown to a cell density of ˜0.2 million cells ml⁻¹ under white LED light and then transferred for either 48 h growth (FIG. 13A) or 60 h growth (FIGS. 13B-13D) under different lighting regimes which included
(1) Continuous far-red light (730 nm),
(2) Continuous red light provided by covering white light with a red filter (LEE filter medium red 027), and
(3) Continuous light at 830 nm supplied with a LED of wavelength 830 nm.
(4) Dark

The carotenoids, 9-cis/all-trans ratio and chlorophyll content of cultures under far red and red light were identical (FIG. 13A). Both far red light and red light increased the 9-cis/all-trans ratio from ˜1.5 to ˜2.0 compared to white light alone (FIG. 13A). Under LED light of wavelength 830 nm applied for 3 days, the cells did not divide, as was also found for cells placed in the dark for 3 days (FIG. 13B). The 9-cis/all-trans β-carotene ratio decreased for cells placed in either the dark or treated with 830 nm light compared to that recorded for the cells at the outset (To) of the experiment (FIG. 13C), and the yield of carotenoids and β-carotene also slightly decreased, albeit not as much as cells placed in the dark (FIG. 13E).FIGS. 14A-14E: Effect of cultivating D. salina under different red/dark cycles. The data show the effect of reducing the duration of red light on (FIG. 14A) Cell density; (FIG. 14B) Cellular content of total carotenoids, (FIG. 14C) Carotenoids/Chlorophyll ratio, (FIG. 14D) cellular content of all-trans β-carotene, (FIG. 14E) 9-cis/all-trans β-carotene ratio.

Cultures of D. salina were grown to a cell density of ˜0.2 million cells ml⁻¹ under white LED light and then transferred into red LED light growth cycles of different duration, which were maintained for 6 days. The light intensity of red LED light was set at 500 μmol m⁻² s⁻¹. The cycles were as follows:
(1) 10 min red LED on, 110 min off (8% cycle time with light)
(2) 20 min red LED on 100 min off (17% cycle time with light)
(3) 10 min red LED on, 50 min off (17% cycle time with light),
(4) 20 min red LED on, 40 min off, (33% cycle time with light),
(5) 30 min red LED on 30 min off (50% cycle time with light),
(6) Continuous red LED light.

FIGS. 15A-15E show the cellular content of (FIG. 15A) phytoene, (FIG. 15B) 9-cis β-carotene, (FIG. 15C) all-trans β-carotene, (FIG. 15D) total β-carotene and (FIG. 15E) 9-cis/all-trans β-carotene ratio in D. salina cultures treated at 25° C. for 48 h with different concentrations of the phytoene desaturase inhibitor herbicide norflurazon (5 and 50 μM) under white or red LED light at 200 μmol m⁻² s⁻¹. Coloured carotenoids and phytoene contents were determined after separation using HPLC as before. When phytoene desaturase was inhibited, phytoene accumulated and a significantly higher amount of phytoene was produced under red light compared to white light (FIG. 15A). Furthermore, in red light 9-cis β-carotene increased (FIG. 15B) at the expense of all-trans β-carotene (FIG. 15C) which was converted to 9-cis β-carotene while no more all-trans β-carotene was synthesized (FIG. 15D), resulting in even higher 9-cis/all-trans β-carotene ratio, more than double the ratio determined in white light (white light, 1.8; red light, 3.9) (FIG. 15E).

FIGS. 16A-16J show the effect of cultivation of D. salina in the presence of chlorpropham.

(FIGS. 16A-16D): The effect of increasing concentrations of chlorpropham on (i) cellular content of phytoene; (ii) phytoene yield; (iii) cellular content of β-carotene; and (iv) total carotenoids.

(FIGS. 16E-16F): The effect of increasing white LED light intensity on phytoene production.

(FIGS. 16G-16H): The effect of red LED light (100-200 μmol m⁻² s⁻¹) on phytoene production.

(FIGS. 16I-16J): The effect of red LED light of increasing light intensity on phytoene production.

Chlorpropham stock solution of 1 M was added to cultures of D. salina to different final concentrations (0, 0.1, 1, 10, 20, 50 and 100 μM) and cultures were maintained in an incubator at 25° C. Carotenoids profile was analysed for each culture by HPLC. For (FIGS. 16A-16D), cultures were maintained under continuously applied white LED light (˜200 μmol m⁻² s⁻¹) with different concentrations of chloropropham as shown. For (FIGS. 16E-16F) cultures were maintained in the presence of 20 μM chlorpropham, but under different intensities of continuously applied white LED light (50, 100, 200, 500, 1000 and 1500 μmol m⁻² s⁻¹) as shown. For (FIGS. 16G-16H), cultures were maintained under continuously applied red LED light at 100-200 μmol m⁻² s⁻¹ in the presence of either 10 μM or 20 μM chlorpropham for 6 days. For (FIGS. 16I-16J) cultures were maintained under continuously applied red LED lights at different light intensities (200, 500, and 1000 μmol m⁻² s⁻¹) for 48 hours in the presence of 20 μM chlorpropham.

The optimal concentration of chlorpropham for phytoene production was between 10-50 μM.

After 6-days cultivation in white LED light in the presence of 20 μM chlorpropham, the phytoene content in cells increased ca. 50-fold compared to that in untreated cells (untreated cells: 0.55±0.01 pg cell⁻¹, treated cells 25.76±1.58 pg cell⁻¹) whilst the final phytoene concentration in the cultures increased 10-fold (untreated cultures 0.35±0.01 mg L⁻¹; treated 3.55±0.11 mg L⁻¹). With increasing light intensity of applied white light, phytoene content per cell and yield increased: after just 4 days' cultivation, the phytoene content reached above 30 pg cell⁻¹ under 1500 μmol m⁻² s⁻¹, giving a yield of 8.2 mg L⁻¹. Under red light, cultures had higher phytoene contents than cultures maintained under white light with the same concentration of chlorpropham treatment.

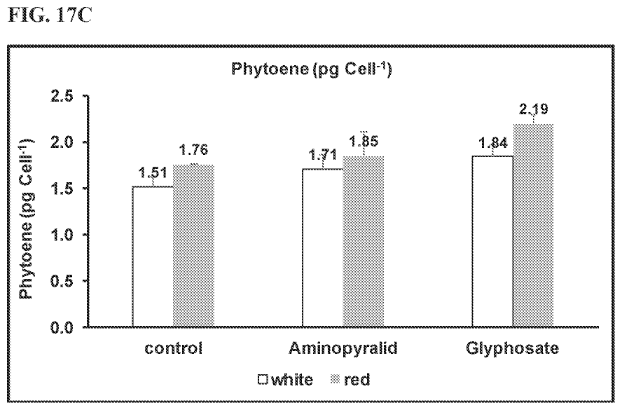

FIGS. 17A-17C show the effect of cultivation of D. salina in the presence of the herbicides aminopyralid, carbetamide, and chlorsulfuron (cell division inhibitors), and glyphosate (phytochrome inhibitor). All herbicides tested increased the content of phytoene per cell and the contents were further increased when cultures were maintained under red light.

(FIGS. 17A-17B): Effect of increasing concentrations of herbicides on cellular content of (i) phytoene; (ii) phytoene yield, under continuous white light.

(FIG. 17C): Effect of red light applied to cultures of D. salina treated with either 50 μM aminopyralid or 50 μM glyphosate as representative cell division or phytochrome inhibitors respectively.

Cultures were maintained at 25° C. under continuous white or red LED light at ˜200 μmol m⁻² s⁻¹ and carotenoid contents determined daily by HPLC as before.

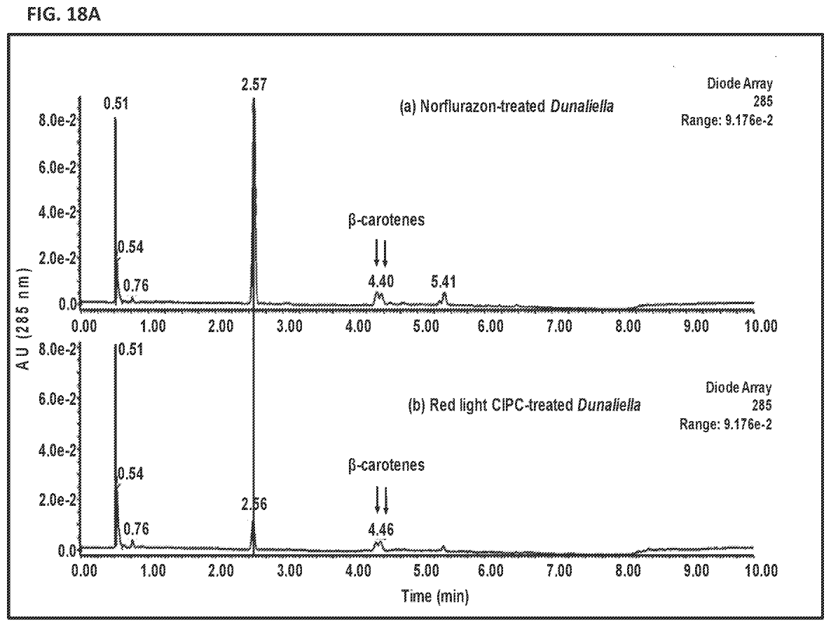

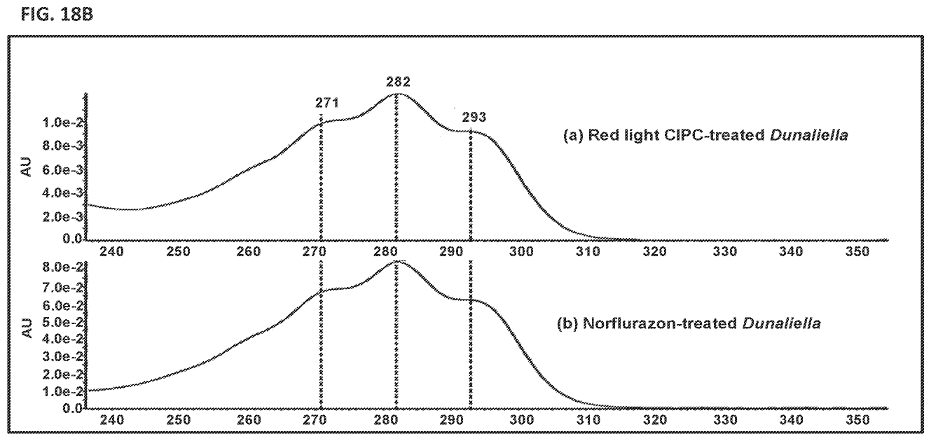

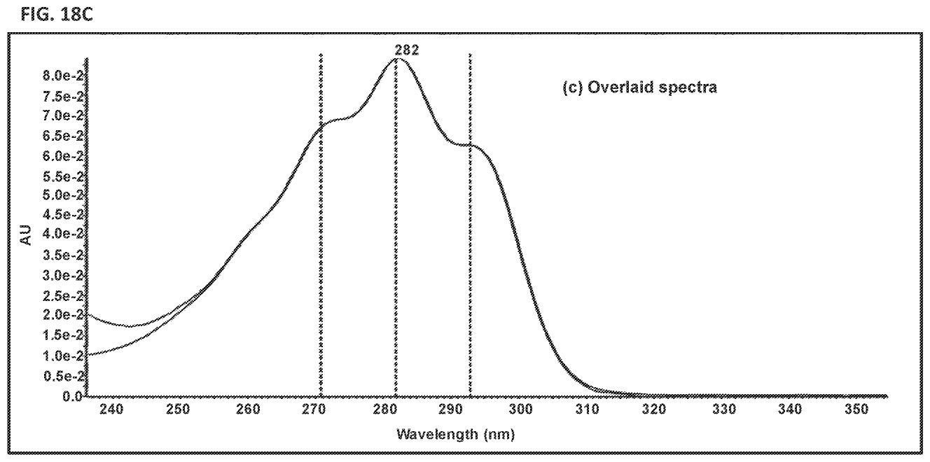

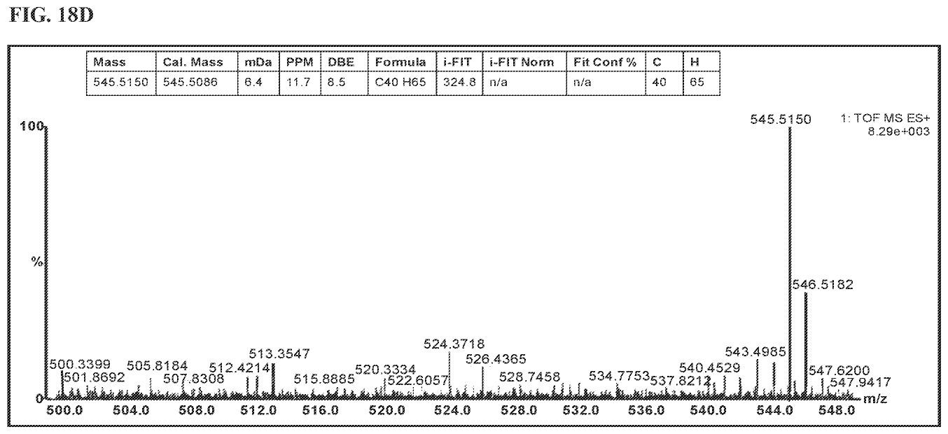

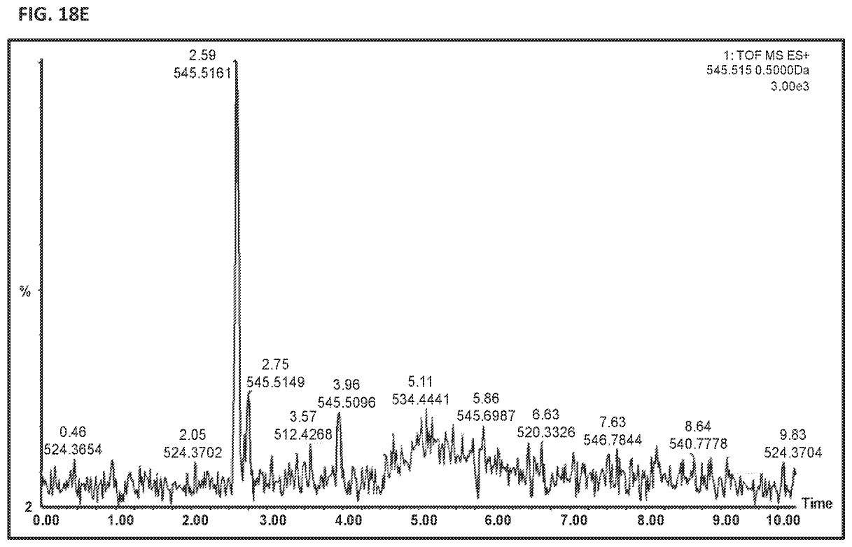

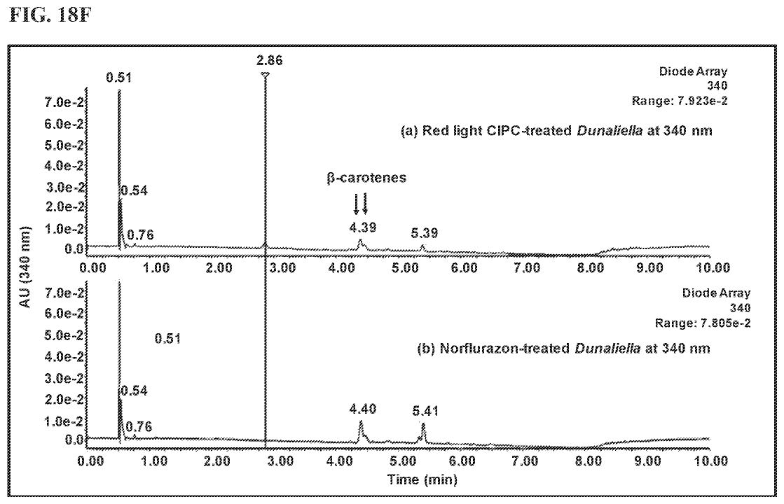

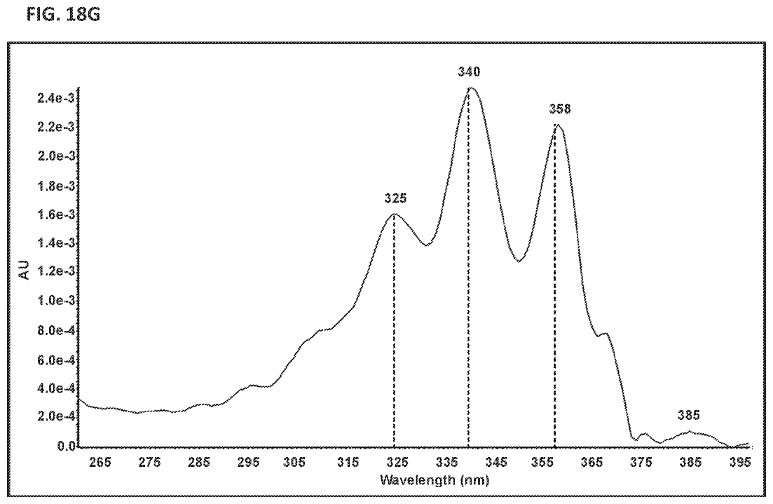

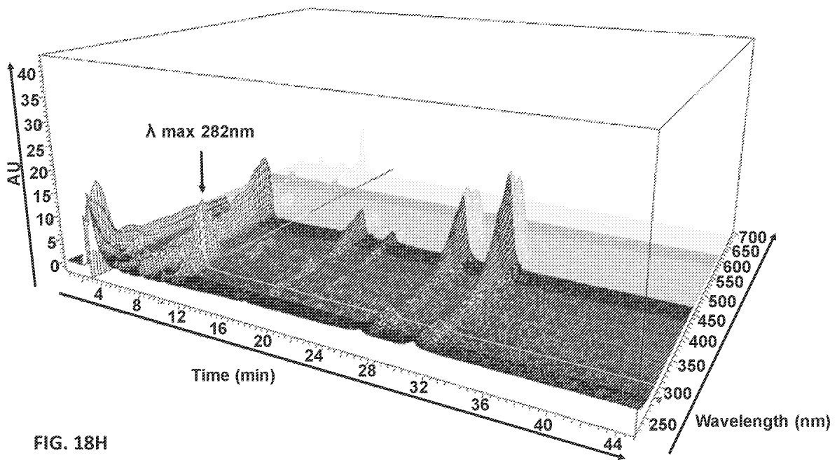

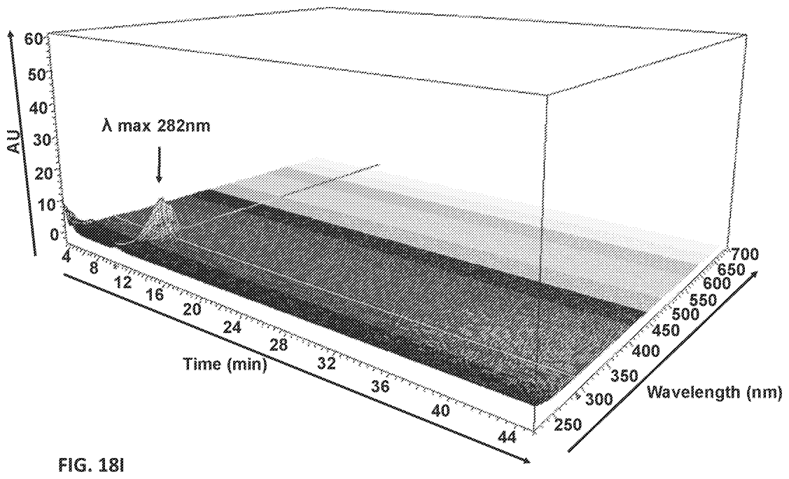

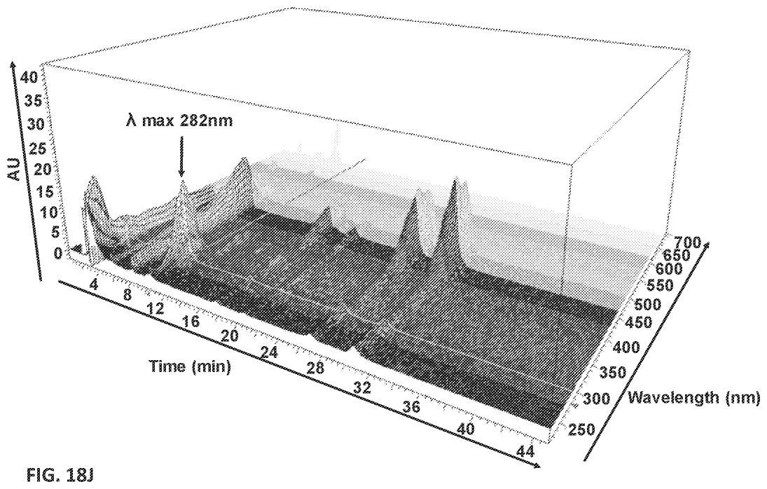

FIGS. 18A-18J provides data to substantiate the identity of phytoene and phytofluene in cultures of D. salina.

Samples were extracted using absolute ethanol and extracts were analysed using a YMC30 250×4.9 mm I.D S-5μ HPLC column with DAD at 25° C., and isocratic elution with 80% methanol: 20% MTBE, flow rate of 1 mL min⁻¹, pressure of 90 bar. Alternatively they were analysed using a Waters Acquity UPCC (Waters, UK) instrument fitted with a Diode Array Detector and connected to a Synapt G2 HDMS (Waters, UK). The Synapt G2 was fitted with an electrospray source, and operated in positive ion mode over a mass range of 50-800 m/z units. Wavelength-dependent absorption was measured using the DAD, and operating in the wavelength range 200-700 nm. Phytoene was separated using an Acquity UPLC HSS C18 SB, 3.0×100 mm, 1.8 μm particle size, inlet conditions: scCO₂ (FIG. 18A); Methanol+0.1% formic acid (v/v) (FIG. 18B); Make-up solvent: Methanol+0.1% formic acid (v/v). Processing was carried out using MassLynx v4.1.

| Time | Flow (ml/min) | % A | % B |
|---|---|---|---|
| Initial | 1.5 | 95.0 | 5.0 |
| 5.00 | 1.5 | 75.0 | 25.0 |
| 5.10 | 1.5 | 50.0 | 50.0 |
| 6.00 | 1.5 | 50.0 | 50.0 |
| 8.00 | 1.5 | 95.0 | 5.0 |
| 10.00 | 1.5 | 95.0 | 5.0 |

FIG. 18A: UPC2 chromatogram (detection 285 nm) for (a) Norflurazon-treated cultures and (b) chlorpropham-treated cultures of D. salina.

FIGS. 18B-18C: Spectral properties of peaks RT of 2.57 min and 2.56 min in samples (a) and (b) respectively and overlay of the spectra. λmax at 282 nm, 293 nm and 271 nm correspond to published λmax values for phytoene.

FIG. 18D: Elemental Composition Analysis of peak at RT=2.57 min.

FIG. 18E: Extracted Ion Chromatogram for m/z=(545.5±0.5) Da for peak at RT=2.57 min.

FIG. 18F: UPC2 chromatogram (detection 340 nm) for (a) chlorpropham-treated cultures and (b) Norflurazon-treated cultures of D. salina.

FIG. 18G: Spectral properties of peak RT of 2.86 min for chlorpropham-treated cultures

FIG. 18H: 3D chromatogram over 220-700 nm of carotenoids extract from D. salina cultures under red light with 20 μM chlorpropham, obtained after separation by HPLC.

FIG. 18I: 3D chromatogram over 220-700 nm of 78903 SIGMA (E/Z)-Phytoene mixture of isomers, ≥95% (HPLC), obtained after separation by HPLC.

FIG. 18J: 3D chromatogram over 220-700 nm of carotenoids extract from D. salina cultures under red light with 20 μM chlorpropham, with a spike of phytoene standard.

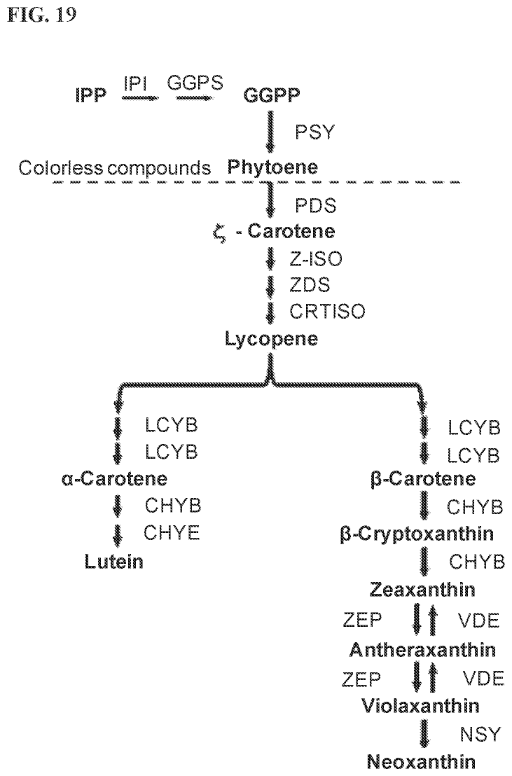

FIG. 19 depicts the carotenoid pathway.

TABLE 1
Effect of different light treatments on the culture concentration of carotenoids and the ratio
of 9-cis and all-trans β-carotene in Dunaliella salina when cultivated to early orange
phase until light treatment (T0) and then subjected to different light treatments for 48 hours.
All conditions as described in FIG. 2A-2C. Data were calculated mean values ± standard
deviations. Each light condition was set up at least in triplicate. Red light whether applied
with LED or filters increased yield of carotenes and the ratio of 9-cis:all-trans β-carotene.

| | | Concentration (μg/ml) | | | | 9-cis:all- |
|---|---|---|---|---|---|---|
| | Cell | All-trans | | All-trans | 9-cis | trans β- |
| Light | density | α- | | β- | β- | carotene |
| treatment | (×10⁶/ml) | carotene | Phytoene | carotene | carotene | ratio |
| Time 0 | 0.22 ± 0.01 | 0.14 ± 0.01 | 0.08 ± 0.01 | 2.12 ± 0.10 | 2.16 ± 0.08 | 1.02 ± 0.09 |
| White | 0.40 ± 0.02 | 0.31 ± 0.03 | 0.88 ± 0.11 | 5.35 ± 0.58 | 5.01 ± 0.21 | 0.95 ± 0.14 |
| Red | 0.37 ± 0.06 | 0.27 ± 0.02 | 1.04 ± 0.06 | 4.02 ± 0.40 | 5.76 ± 0.97 | 1.44 ± 0.27 |
| Blue | 0.38 ± 0.03 | 0.19 ± 0.01 | 0.67 ± 0.06 | 4.72 ± 0.25 | 2.95 ± 0.21 | 0.63 ± 0.04 |
| White + | 0.38 ± 0.01 | 0.26 ± 0.02 | 0.64 ± 0.04 | 3.78 ± 0.44 | 6.60 ± 0.67 | 1.75 ± 0.03 |
| filter26 | | | | | | |
| White + | 0.41 ± 0.01 | 0.24 ± 0.05 | 0.59 ± 0.14 | 3.93 ± 0.34 | 7.07 ± 0.58 | 1.80 ± 0.16 |
| filter27 | | | | | | |
| White + | 0.36 ± 0.02 | 0.16 ± 0.02 | 0.27 ± 0.03 | 2.89 ± 0.32 | 4.68 ± 0.39 | 1.62 ± 0.06 |
| filter787 | | | | | | |

TABLE 2
Effect of different light treatments on culture concentration of carotenoids
and the ratio of 9-cis and all-trans β-carotene in Dunaliella salina when
cultivated to mid-log phase of growth until light treatment (T0) and then subjected
to different light treatments. All conditions as described in FIG. 4A-4B. Data
were calculated mean values ± standard deviations. Each light condition
was set up at least in triplicate. Red LED light increased the entire pathway
of carotene production since contents of all carotenoids increased in parallel
with the previously reported increases in carotene content described above.

| | | Concentration (μg/ml) | | | | |
|---|---|---|---|---|---|---|
| | | All-trans | | All-trans | 9-cis | 9-cis:all- |
| | | α- | | β- | β-c | trans |
| Time | Light | carotene | Phytoene | carotene | arotene | ratio |
| Control | White | 0.07 ± 0.03 | — | 0.63 ± 0.16 | 0.45 ± 0.08 | 0.71 |
| 0 | Dark | 0.02 ± 0.00 | 0.07 ± 0.01 | 0.51 ± 0.08 | 0.30 ± 0.11 | 0.59 |
| 0-24 h | Red | 0.05 ± 0.00 | 0.16 ± 0.02 | 0.60 ± 0.07 | 0.73 ± 0.15 | 1.22 |
| 24-48 h | Red | 0.12 ± 0.04 | 0.41 ± 0.03 | 1.12 ± 0.24 | 1.77 ± 0.26 | 1.58 |
| | Blue | 0.06 ± 0.00 | 0.14 ± 0.03 | 1.03 ± 0.07 | 0.53 ± 0.02 | 0.51 |
| | Mix | 0.10 ± 0.00 | 0.21 ± 0.02 | 1.12 ± 0.03 | 0.84 ± 0.02 | 0.75 |
| | Dark | 0.06 ± 0.00 | 0.16 ± 0.01 | 0.79 ± 0.15 | 0.82 ± 0.11 | 1.04 |

TABLE 3
Cellular content of carotenoids in Dunaliella salina cells treated with either
continuous blue or red LED light at three different light intensities of 200, 500 and
1000 μmol m⁻² s⁻¹ for 48 hours. All conditions as described in FIG. 6A-6E.

| | | Cellular content (pg cell⁻¹) | | | | 9-cis:all- |
|---|---|---|---|---|---|---|
| | Intensity | All-trans | | All-trans | 9-cis | trans |
| | (μmol | α- | | β- | β- | β-carotene |
| Light | m⁻² s⁻¹) | carotene | Phytoene | carotene | carotene | ratio |
| Red | 200 | 0.68 ± 0.10 | 1.49 ± 0.26 | 5.93 ± 0.07 | 10.04 ± 1.38 | 1.69 |
| | 500 | 0.84 ± 0.13 | 2.10 ± 0.24 | 6.80 ± 0.01 | 11.93 ± 1.59 | 1.75 |
| | 1000 | 0.66 ± 0.09 | 2.28 ± 0.45 | 6.21 ± 0.60 | 9.75 ± 1.09 | 1.57 |
| Blue | 200 | 0.34 ± 0.06 | 0.79 ± 0.19 | 5.51 ± 1.40 | 3.98 ± 0.96 | 0.72 |
| | 500 | 0.47 ± 0.08 | 1.27 ± 0.25 | 7.68 ± 1.66 | 4.79 ± 0.85 | 0.62 |
| | 1000 | 0.64 ± 0.09 | 2.23 ± 0.41 | 10.27 ± 1.23 | 10.03 ± 1.46 | 0.98 |

It can be seen from the data presented in Tables 1 to 3, and FIGS. 1 to 8, that exposure of Dunaliella salina to red light results in a significant increase in the content of total carotenoids, particularly an increase in the content of 9-cis-β-carotene, and a significant increase in the ratio of 9-cis to all-trans β-carotene. The data also show that exposure of Dunaliella salina to red light also increased phytoene (a colourless carotenoid) and α-carotene.

Table 4 provides a comparison of the contents of carotenoids for cultures of D. salina cultivated for 48 h under either white LED light (A) or red LED light (B) in the presence of either Norflurazon or chlorpropham (C1PO compared to cultivation without herbicide Both white and red LED lights were applied at 200 pmol m⁻² s⁻¹ continuously for 48 h after adding the herbicides Red light more than doubled the vield of phv toene in either CIPC or norflurazon-treated cultures increased the content of total carotenoids, and increased the ratio of 9-cts all-trans β-carotene

| | | | | | | | | | | |
|---|---|---|---|---|---|---|---|---|---|---|
| | | | | | | | | Total | phytoene + | |
| carot- | | | All-trans | 9-cis | | All-trans | | carot- | phyto- | 9cis |
| enoids | | Phyto- | □- | □- | zea- | □- | | enoids | fluene | of |
| (pg cell⁻¹) | Phytoene | fluene | carotene | carotene | xanthin | carotene | lutein | (sum) | of total | total |

(A) Cultivation under white LED light (48 h treatment)

| | | | | | | | | | | |
|---|---|---|---|---|---|---|---|---|---|---|
| No herbicide | 0.79 ± 0.04 | 0.07 ± 0.00 | 6.46 ± 0.10 | 10.73 ± 0.22 | 0.90 ± 0.08 | 0.44 ± 0.02 | 0.66 ± 0.01 | 20.05 | 4% | 54% |
| chlor- | 4.41 ± 0.65 | 0.67 ± 0.09 | 10.56 ± 1.57 | 13.35 ± 1.88 | 0.84 ± 0.09 | 0.33 ± 0.02 | 0.83 ± 0.13 | 30.99 | 16% | 43% |
| propham | | | | | | | | | | |
| (10 μM) | | | | | | | | | | |
| Norflurazon | 7.07 ± 0.36 | 0.01 ± 0.00 | 4.28 ± 0.18 | 7.65 ± 0.67 | 0.80 ± 0.07 | 0.27 ± 0.01 | 0.52 ± 0.02 | 20.6 | 34% | 37% |
| (5 □M) | | | | | | | | | | |

(B) Cultivation under red light (48 h treatment)

| | | | | | | | | | | |
|---|---|---|---|---|---|---|---|---|---|---|
| No herbicide | 0.98 ± 0.02 | 0.13 ± 0.00 | 5.58 ± 0.60 | 13.40 ± 1.25 | 1.17 ± 0.12 | 0.41 ± 0.05 | 0.69 ± 0.04 | 22.36 | 5% | 60% |
| chlor- | 10.56 ± 1.53 | 1.58 ± 0.17 | 7.91 ± 0.38 | 14.35 ± 1.24 | 1.05 ± 0.06 | 0.51 ± 0.02 | 0.74 ± 0.05 | 36.7 | 33% | 39% |
| propham | | | | | | | | | | |
| (10 μM) | | | | | | | | | | |
| Norflurazon | 14.42 ± 0.95 | 0.02 ± 0.00 | 3.09 ± 0.30 | 11.89 ± 0.83 | 1.12 ± 0.06 | 0.39 ± 0.01 | 0.62 ± 0.02 | 31.55 | 46% | 38% |
| (5 □M) | | | | | | | | | | |

Table 5 shows the contents of carotenoids produced for cultures of D. salina cultivated for 96 h and 144 h under red light of varying intensities and different illumination time, with the application of chlorpropham. Data for diflufenican are also shown.

| | | | | | | | | Total | phytoene + | |
|---|---|---|---|---|---|---|---|---|---|---|
| carot- | | | All-trans | 9-cis | | All-trans | | carot- | phyto- | |
| enoids | | Phyto- | β- | β- | zea- | α- | | enoids | fluene | 9cis of |
| (pg cell⁻¹) | Phytoene | fluene | carotene | carotene | xanthin | carotene | lutein | (sum) | of total | total |
| chlor- | 30.62 ± 1.31 | 1.87 ± 0.12 | 17.34 ± 0.97 | 6.96 ± 0.74 | 0.77 ± 0.02 | 0.44 ± 0.07 | 0.61 ± 0.03 | 58.61 | 55% | 12% |
| propham | | | | | | | | | | |
| 1500 | | | | | | | | | | |
| μmol m⁻² | | | | | | | | | | |
| s⁻¹ 96 h | | | | | | | | | | |
| chlor- | 51.88 ± 4.53 | 3.02 ± 0.29 | 18.34 ± 1.45 | 31.3 ± 2.49 | 1.33 ± 0.10 | 1.4 ± 0.13 | 1.21 ± 0.09 | 108.48 | 51% | 29% |
| propham | | | | | | | | | | |
| 200 | | | | | | | | | | |
| μmolm⁻² | | | | | | | | | | |
| s⁻¹ 144 h | | | | | | | | | | |
| Diflufenican | 12.78 ± 0.40 | 0.01 ± 0.00 | 2.98 ± 0.11 | 10.84 ± 0.19 | 1.13 ± 0.06 | 0.34 ± 0.00 | 0.56 ± 0.01 | 28.64 | 45% | 38% |
| 200 | | | | | | | | | | |
| μmol m⁻² | | | | | | | | | | |
| s⁻¹ 48 h | | | | | | | | | | |

1. A process for the preparation of a Dunaliella alga or extract thereof, comprising the steps:
a) cultivating the Dunaliella alga under white light; and subsequently;
b) exposing the Dunaliella alga to light of wavelength 500-1000 nm, or 500-700 nm or 700-1000 nm; and/or eliminating light of wavelength less than 500 nm (blue light).

2. The process according to claim 1, comprising the steps:
a) cultivating the Dunaliella alga under white light; subsequently;
b) exposing the Dunaliella alga to light of wavelength 500-1000 nm, or 500-700 nm or 700-1000 nm; and/or eliminating light of wavelength less than 500 nm (blue light); and
applying a herbicide to the Dunaliella alga during step a) and/or step b).

3. The process according to claim 2, wherein the herbicide is selected from amino acid synthesis inhibitors, growth regulators, nitrogen metabolism inhibitors, pigment inhibitors, seedling root growth inhibitors, seedling shoot growth inhibitors, cell wall synthesis inhibitors, mitosis microtubule organisation inhibitors, and combinations thereof.

4. The process according to claim 2, wherein the herbicide is selected from norflurazon, diflufenican, picolinafen, beflubutamid, fluridone, flurochloridone, flurtamone, chlorpropham, propham, carbetamide, and combinations thereof.

5. A process for the preparation of a Dunaliella alga, comprising applying the Dunaliella alga with a herbicide selected from the group consisting of amino acid synthesis inhibitors, growth regulators, nitrogen metabolism inhibitor, pigment inhibitors (excluding phytoene desaturase inhibitors), seedling root growth inhibitors, seedling shoot growth inhibitors, cell wall synthesis inhibitors, mitosis microtubule organisation inhibitors, and combinations thereof.

6. The process according to claim 5, wherein the herbicide is selected from chlorpropham, propham, carbetamide, and combinations thereof.
